(19) United States
(12) Patent Application Publication
(10) Pub. No.: US 2008/0076926 A1
(43) Pub. Date: Mar. 27, 2008

(54) Process for the preparation of 4-hydroxythieno[2,3-b]pyridine-5-carbonitriles

(76) Inventors: Diane Harris Boschelli, New City, NY (US); Lawrence Nathan Tumey, New Windsor, NY (US); Biqi Wu, Nanuet, NY (US)

Correspondence Address: KIRKPATRICK & LOCKHART PRESTON GATES ELLIS LLP;(FORMERLY KIRKPATRICK&LOCKHART NICHOLSON GRAHAM), STATE STREET FINANCIAL CENTER, ONE LINCOLN STREET, BOSTON, MA, 02111-2950, US

(21) Appl. No.: 11/903,918

(22) Filed: Sep. 25, 2007

Related U.S. Application Data
(60) Provisional application No. 60/847,334, filed on Sep. 26, 2006.
(60) Provisional application No. 60/956,253, filed on Aug. 16, 2007.

(51) Int. Cl. C07D 495/04

(52) U.S. Cl. 546/114

(57) ABSTRACT

A process for the preparation of 4-hydroxythieno[2,3-b]pyridine-5-carbonitriles, which can be useful for the preparation of protein kinase inhibitors, is provided.

### CROSS-REFERENCE TO RELATED APPLICATIONS

This application claims the benefit under 35 U.S.C. § 119(e) of U.S. Provisional Patent Application Ser. Nos. 60/847,334 and 60/956,253, filed on Sep. 26, 2006, and Aug. 16, 2007, respectively, the entire disclosures of which are incorporated by reference herein.

### INTRODUCTION

The present teachings relate to a method for preparing 4-hydroxy-thieno-[2,3-b]-pyridine-5-carbonitriles, which can be used for preparing compounds that can be used as protein kinase inhibitors. The present teachings also relate to a method for preparing 4-hydroxy-thieno[2,3-b]pyridine-5-carbonitriles and converting them into compounds that can be used as protein kinase inhibitors.

Protein kinases are enzymes that catalyze the transfer of a phosphate group from adenosine triphosphate (ATP) to an amino acid residue, such as tyrosine, serine, threonine, or histidine, on a protein. Regulation of these protein kinases is essential for the control of a wide variety of cellular events including proliferation and migration. A large number of diseases are associated with these kinase-mediated abnormal cellular events including various inflammatory diseases and autoimmune diseases such as asthma, psoriasis, arthritis, rheumatoid arthritis, osteoarthritis, joint inflammation, multiple sclerosis, diabetes including type II diabetes, and inflammatory bowel diseases such as Crohn's disease and colitis (Kim, J. et al. (2004), J. Clin. Invest., 114: 823-827; Schmitz-Peiffer, C. et al. (2005), Drug Discov Today, 2(2): 105-110; Salek-Ardakani, S. et al. (2005), J. Immunol., 175: 7635-7641; Healy. A. et al. (2006), J. Immunol., 177: 1886-1893; and Tan, S-L. (2006), J. Immunol., 176: 2872-2879).

One class of serine/threonine kinases is the protein kinase C (PKC) family. This group of kinases consists of 10 members that share sequence and structural homology. The PKCs are divided into 3 groups and include the classic, the novel, and the atypical isoforms. The theta isoform (PKCθ) is a member of the novel calcium-independent class of PKCs (Baier, G. et al. (1993), J. Biol. Chem., 268: 4997-5004). PKCθ is highly expressed in T cells (Mischak, H. et al. (1993), FEBS Lett., 326: 51-5), with some expression reported in mast cells (Liu, Y. et al. (2001), J. Leukoc. Biol., 69: 831-40), endothelial cells (Mattila, P. et al. (1994), Life Sci., 55: 1253-60), and skeletal muscle (Baier, G. et al. (1994), Eur. J. Biochem., 225: 195-203). It has been shown that PKCθ plays an essential role in T cell receptor (TCR)-mediated signaling (Tan, S. L. et al. (2003), Biochem. J., 376: 545-52). Specifically, it has been observed that inhibiting PKCθ signal transduction, as demonstrated with two independent PKCθ knockout mouse lines, will result in defects in T cell activation and interleukin-2 (IL-2) production (Sun, Z. et al. (2000), Nature, 404: 402-7; Pfeifhofer, C. et al. (2003), J. Exp. Med., 197: 1525-35). It also has been shown that PKCθ-deficient mice show impaired pulmonary inflammation and airway hyperresponsiveness (AHR) in a Th2-dependent murine asthma model, with no defects in viral clearance and Th1-dependent cytotoxic T cell function (Berg-Brown, N. N. et al. (2004), J. Exp. Med., 199: 743-52; Marsland, B. J. et al. (2004), J. Exp. Med., 200: 181-9). The impaired Th2 cell responses result in reduced levels of interleukin-4 (IL-4) and immunoglobulin E (IgE), contributing to the AHR and inflammatory pathophysiology.

Evidence also exists that PKCθ participates in the IgE receptor (FceRI)-mediated response of mast cells (Liu, Y. et al. (2001), J. Leukoc. Biol., 69: 831-840). In human-cultured mast cells (HCMC), it has been demonstrated that PKC kinase activity rapidly localizes (in less than five minutes) to the membrane following FceRI cross-linking (Kimata, M. et al. (1999), Biochem. Biophys. Res. Commun., 257(3): 895-900). A recent study examining in vitro activation of bone marrow mast cells (BMMCs) derived from wild-type and PKCθ-deficient mice shows that upon FceRI cross-linking, BMMCs from PKCθ-deficient mice produced reduced levels of interleukin-6 (IL-6), tumor necrosis factor-alpha (TNFα), and interleukin-13 (IL-13) in comparison with BMMCs from wild-type mice, suggesting a potential role for PKCθ in mast cell cytokine production in addition to T cell activation (Ciarletta, A. B. et al. (2005), poster presentation at the 2005 American Thorasic Society International Conference).

Other serine/threonine kinases include those of the mitogen-activated protein kinase (MAPK) pathway which consists of the MAP kinase kinases (MAPKK) (e.g., mek and their substrates) and the MAP kinases (MAPK) (e.g., erk). Members of the raf family of kinases phosphorylate residues on mek. The cyclin-dependent kinases (cdks), including cdc2/cyclin B, cdk2/cyclin A, cdk2/cyclin E and cdk4/cyclin D, and others, are serine/threonine kinases that regulate mammalian cell division. Additional serine/threonine kinases include the protein kinases A and B. These kinases, known as PKA or cyclic AMP-dependent protein kinase and PKB (Akt), play key roles in signal transduction pathways.

Tyrosine kinases (TKs) are divided into two classes: the non-transmembrane TKs and transmembrane growth factor receptor TKs (RTKs). Growth factors, such as epidermal growth factor (EGF), bind to the extracellular domain of their partner RTK on the cell surface which activates the RTK, initiating a signal transduction cascade that controls a wide variety of cellular responses. In addition to EGF, there are several other RTKs including FGFr (the receptor for fibroblast growth factor (FGF)); flk-1 (also known as KDR, and flt-1, the receptors for vascular endothelial growth factor (VEGF)); and PDGFr (the receptor for platelet derived growth factor (PDGF)). Other RTKs include tie-1 and tie-2, colony stimulating factor receptor, the nerve growth factor receptor, and the insulin-like growth factor receptor. In addition to the RTKs there is another family of TKs termed the cytoplasmic protein or non-receptor TKs. The cytoplasmic protein TKs have intrinsic kinase activity, are present in the cytoplasm and nucleus, and participate in diverse signaling pathways. There is a large number of non-receptor TKs including Abl, Jak, Fak, Syk, Zap-70 and Csk and also the Src family of kinases (SFKs) which includes Src, Lck, Lyn, Fyn, Yes and others.

One group of protein kinase inhibitors that can be prepared using the methods of the present teachings are described in U.S. patent application Ser. No. 11/527,996, published as U.S. Patent Application Publication No. 2007/0082880 A1, the entire disclosure of which is incorporated by reference herein. Another group of protein kinase inhibitors that can be prepared using the methods of the present teachings are described in U.S. patent application Ser. No. 10/719,359, issued as U.S. Pat. No. 6,987,116 B2, the entire disclosure of which is incorporated by reference herein.

Given the large number of diseases that have been associated with protein kinases, there is a continuing need in the art for new methods for preparing protein kinase inhibitors. For example, 4-chloro-2-iodothieno[2,3-b]pyridine-5-carbonitrile is a versatile intermediate in the synthesis of substituted thieno[2,3-b]pyridine-5-carbonitriles. While various synthetic schemes have been used to prepare this intermediate (see, e.g., Boschelli, D. H. et al. (2004), J. Med. Chem., 47(27): 6666-68), alternative synthetic methods that are readily scalable and provide greater diversification are desired in the art.

### SUMMARY

One aspect of the present teachings provides a method for preparing a compound of formula VI or a tautomer thereof: 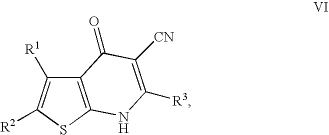
wherein R¹, R², and R³ are as defined herein.

Another aspect of the present teachings provides a method for preparing a compound of formula VI or a tautomer thereof, and converting it into a compound of formula XI: 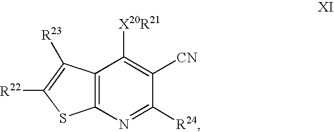
or an N-oxide, sulfoxide, or sulfone derivative thereof, wherein R²¹-R²⁴ and X²⁰ are as defined herein.

Another aspect of the present teachings provides a method for preparing a compound of formula VI or a tautomer thereof, and converting it into a compound of formula XII: 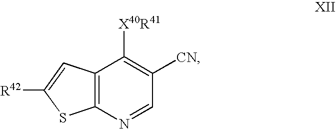
or a sulfoxide or sulfone derivative thereof, wherein R⁴¹-R⁴² and X⁴⁰ are as defined herein.

The foregoing, and other features and advantages of the present teachings will be more fully understood from the following description, examples, and claims.

### DETAILED DESCRIPTION

Throughout the description, where compositions are described as having, including, or comprising specific components, or where processes are described as having, including, or comprising specific process steps, it is contemplated that compositions of the present teachings also consist essentially of, or consist of, the recited components, and that the processes of the present teachings also consist essentially of, or consist of, the recited process steps.

In the application, where an element or component is said to be included in and/or selected from a list of recited elements or components, it should be understood that the element or component can be any one of the recited elements or components and can be selected from a group consisting of two or more of the recited elements or components. Further, it should be understood that elements and/or features of a compound, a composition, or a method described herein can be combined in a variety of ways without departing from the spirit and scope of the present teachings, whether explicit or implicit herein.

The terms “include,” “includes,” “including,” “have,” “has,” or “having” should be understood as open-ended and non-limiting unless specifically stated otherwise.

The use of the singular herein includes the plural (and vice versa) unless specifically stated otherwise. In addition, where the use of the term “about” is before a quantitative value, the present teachings also include the specific quantitative value itself, unless specifically stated otherwise.

It should be understood that the order of steps or order for performing certain actions is immaterial so long as the present teachings remain operable. Moreover, two or more steps or actions may be conducted simultaneously.

As used herein, the term “about” refers to a ±5% variation from the nominal value.

As used herein, “tautomers” refer to structural isomers that can be interconvertible by the migration of a proton and the switch of adjacent single bond/s and double bond/s. For example, a compound of formula VI can have a tautomer of the formula: 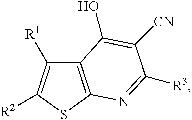
wherein R¹, R², and R³ are as defined herein. It will be understood that a tautomeric compound will generally exist simultaneously in the two tautomeric forms (for example the “keto” form and the “enol” form). A tautomeric compound may therefore be described chemically by nomenclature which either describes the “keto” form or the “enol” form. Whichever nomenclature is used, the same compound is intended. Thus, for example, the compound prepared in Example 1 wherein R¹, R², and R³ are all hydrogen is designated therein as 4-hydroxythieno[2,3-b]pyridine-5-carbonitrile which is the “enol” form. The same compound could equally be described by nomenclature reflecting the “keto” form as 4-oxo-4,7-dihydrothieno[2,3-b]pyridine-5-carbonitrile. Similarly, the compound prepared in Example 3 wherein R² is methyl and R¹ and R³ are hydrogen is described therein in terms of the “keto” form as 3-methyl-4-oxo-4,7-dihydrothieno[2,3-b]pyridine-5-carbonitrile. The same compound could equally well be described in terms of the “enol” nomenclature as 3-methyl-4-hydroxythieno[2,3-b]pyridine-5-carbonitrile.

As used herein, “halo” or “halogen” includes fluoro, chloro, bromo, and iodo.

As used herein, the term “alkyl” refers to a straight-chain or branched saturated hydrocarbon group. In some embodiments, an alkyl group can have from 1 to 10 carbon atoms (e.g., from 2 to 6 carbon atoms). Examples of alkyl groups include methyl (Me), ethyl (Et), propyl, isopropyl, butyl, isobutyl, s-butyl, t-butyl, pentyl, isopentyl, neopentyl, hexyl, and the like. Alkyl groups can be specified to have a limited number of carbon atoms, e.g., C₁₋₆ or C₁₋₄.

As used herein, “alkenyl” refers to a straight-chain or branched hydrocarbon group having one or more carbon-carbon double bonds. In some embodiments, an alkenyl group can have from 2 to 10 carbon atoms (e.g., from 2 to 6 carbon atoms). Examples of alkenyl groups include ethenyl, propenyl, butenyl, pentenyl, hexenyl, butadienyl, pentadienyl, hexadienyl, and the like. The one or more carbon-carbon double bonds can be internal (such as in 2-butenyl) or terminal (such as in 1-butenyl).

As used herein, “alkynyl” refers to a straight-chain or branched hydrocarbon group having one or more carbon-carbon triple bonds. In some embodiments, an alkynyl group can have from 2 to 10 carbon atoms (e.g., from 2 to 6 carbon atoms). Examples of alkynyl groups include ethynyl, propynyl, butynyl, pentynyl, and the like. The one or more carbon-carbon triple bonds can be internal (such as in 2-butynyl) or terminal (such as in 1-butynyl).

As used herein, “cycloalkyl” refers to a non-aromatic carbocyclic group including cyclized alkyl, alkenyl, and alkynyl groups. A cycloalkyl group can be monocyclic (e.g., cyclohexyl) or polycyclic (e.g., containing fused, bridged, and/or spiro ring systems), where the carbon atoms can be located inside or outside of the ring system. A cycloalkyl group, as a whole, can have from 3 to 14 ring atoms (e.g., from 3 to 8 carbon atoms for a monocyclic cycloalkyl group and from 7 to 14 carbon atoms for a polycyclic cycloalkyl group). Any suitable ring position of the cycloalkyl group can be covalently linked to the defined chemical structure. Examples of cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcaryl, adamantyl, and spiro[4.5]decanyl groups, as well as their homologs, isomers, and the like.

As used herein, “alkoxy” refers to an —O-alkyl group, an —O-alkenyl group, an —O-alkynyl group, or an —O-cycloalkyl group. In some embodiments, an alkoxy group can have from 1 to 10 carbon atoms (e.g., from 1 to 6 carbon atoms). Examples of alkoxy groups include methoxy, ethoxy, propoxy, isopropoxy, t-butoxy, allyloxy, cyclopropoxy, cyclobutoxy, cyclohexyloxy, and the like.

As used herein, “heteroatom” refers to an atom of any element other than carbon or hydrogen and includes, for example, nitrogen (N), oxygen (O), sulfur (S), phosphorus (P), selenium (Se), and silicon (Si).

As used herein, “cycloheteroalkyl” refers to a non-aromatic cycloalkyl group that contains at least one ring heteroatom selected from O, N, and S, which can be the same or different, and optionally contains one or more double or triple bonds. A cycloheteroalkyl group, as a whole, can have, for example, from 3 to 14 ring atoms (e.g., from 3 to 7 ring atoms for a monocyclic cycloheteroalkyl group and from 7 to 14 ring atoms for a polycyclic cycloheteroalkyl group) and can contain from 1 to 5 ring heteroatoms. One or more N or S atoms in a cycloheteroalkyl ring can be oxidized (e.g., morpholine N-oxide, thiomorpholine S-oxide, thiomorpholine S,S-dioxide). Cycloheteroalkyl groups can also contain one or more oxo groups, such as oxopiperidyl, oxooxazolidyl, dioxo-(1H,3H)-pyrimidyl, oxo-2(1H)-pyridyl, and the like. Examples of cycloheteroalkyl groups include morpholinyl, thiomorpholinyl, pyranyl, imidazolidinyl, imidazolinyl, oxazolidinyl, pyrazolidinyl, pyrazolinyl, pyrrolidinyl, pyrrolinyl, tetrahydrofuranyl, tetrahydrothiophenyl, piperidinyl, piperazinyl, and the like.

As used herein, “aryl” refers to an aromatic monocyclic hydrocarbon ring system or a polycyclic ring system in which two or more aromatic hydrocarbon rings are fused (i.e., having a bond in common with) together or at least one aromatic monocyclic hydrocarbon ring is fused to one or more cycloalkyl and/or cycloheteroalkyl rings. An aryl group can have from 6 to 14 carbon atoms in its ring system, which can include multiple fused rings. In some embodiments, a polycyclic aryl group can have from 8 to 14 carbon atoms. Any suitable ring position of the aryl group can be covalently linked to the defined chemical structure. Examples of aryl groups having only aromatic carbocyclic ring(s) include phenyl, 1-naphthyl (bicyclic), 2-naphthyl (bicyclic), anthracenyl (tricyclic), phenanthrenyl (tricyclic), and like groups. Examples of polycyclic ring systems in which at least one aromatic carbocyclic ring is fused to one or more cycloalkyl and/or cycloheteroalkyl rings include benzo derivatives of cyclopentane (e.g., an indanyl group, which is a 5,6-bicyclic cycloalkyl/aromatic ring system), cyclohexane (e.g., a tetrahydronaphthyl group, which is a 6,6-bicyclic cycloalkyl/aromatic ring system), imidazoline (e.g., a benzimidazolinyl group, which is a 5,6-bicyclic cycloheteroalkyl/aromatic ring system), and pyran (e.g., a chromenyl group, which is a 6,6-bicyclic cycloheteroalkyl/aromatic ring system). Other examples of aryl groups include benzodioxanyl, benzodioxolyl, chromanyl, indolinyl, and the like.

As used herein, “heteroaryl” refers to an aromatic monocyclic ring system containing at least 1 ring heteroatom selected from oxygen (O), nitrogen (N), and sulfur (S) or a polycyclic ring system where at least one of the rings present in the ring system is aromatic and contains at least 1 ring heteroatom. When more than one ring heteroatoms are present they can be the same or different. Polycyclic heteroaryl groups include two or more heteroaryl rings fused together and monocyclic heteroaryl rings fused to one or more aryl groups, cycloalkyl groups, and/or cycloheteroalkyl rings. A heteroaryl group, as a whole, can have, for example, from 5 to 14 ring atoms and contain 1-5 ring heteroatoms. The heteroaryl group can be attached to the defined chemical structure at any heteroatom or carbon atom that results in a stable structure. Generally, heteroaryl rings do not contain O—O, S—S, or S—O bonds. However, one or more N or S atoms in a heteroaryl group can be oxidized (e.g., pyridine N-oxide, thiophene S-oxide, thiophene S,S-dioxide). Examples of heteroaryl groups include pyrrolyl, furyl, thienyl, pyridyl, pyrimidyl, pyridazinyl, pyrazinyl, triazolyl, tetrazolyl, pyrazolyl, imidazolyl, isothiazolyl, thiazolyl, thiadiazolyl, isoxazolyl, oxazolyl, oxadiazolyl, indolyl, isoindolyl, benzofuryl, benzothienyl, quinolyl, 2-methylquinolyl, isoquinolyl, quinoxalyl, quinazolyl, benzotriazolyl, benzimidazolyl, benzothiazolyl, benzisothiazolyl, benzisoxazolyl, benzoxadiazolyl, benzoxazolyl, cinnolinyl, 1H-indazolyl, 2H-indazolyl, indolizinyl, isobenzofuyl, naphthyridinyl, phthalazinyl, pteridinyl, purinyl, oxazolopyridinyl, thiazolopyridinyl, imidazopyridinyl, furopyridinyl, thienopyridinyl, pyridopyrimidinyl, pyridopyrazinyl, pyridopyridazinyl, thienothiazolyl, thienoxazolyl, thienoimidazolyl, and the like.

As used herein, “heterocyclic” refers to a cycloheteroalkyl group optionally fused to an aryl group and/or a heteroaryl group, where the cycloheteroalkyl group, the aryl group, and the heteroaryl group are defined herein. A heterocyclic group, as a whole, can have, for example, 3 to 14 ring atoms and contain 1-5 ring heteroatoms. The heterocyclic group can be attached to the defined chemical structure at any heteroatom or carbon atom that results in a stable structure:

As used herein, a “divalent group” is defined as a linking group capable of forming a covalent bond with two other moieties. For example, compounds described herein can include a divalent C₁₋₆ alkyl group (e.g., —(C₁₋₆ alkyl)-), such as, for example, a methylene group.

As used herein, a “base” refers to a chemical species or a molecular entity having an available pair of electrons capable of forming a covalent bond with a proton or with a vacant orbital of some other species. Examples of bases include triethylamine, diisopropylethylamine, pyridine, diazobicyclo[2.2.3]undecene, sodium hydride, piperidine, dimethylaminopyridine, potassium tert-butoxide, sodium hydroxide, sodium carbonate, sodium bicarbonate, and the like.

At various places in the present application temperatures are disclosed in ranges. It is specifically intended that the description includes narrower ranges of temperatures within such ranges, as well as the maximum and minimum temperatures embracing such range of temperatures.

At various places in the present application substituents of compounds of the present teachings are disclosed in groups or in ranges. It is specifically intended that the description includes each and every individual subcombination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl groups.

Compounds described herein can contain an asymmetric atom (also referred as a chiral center), and some of the compounds can contain one or more asymmetric atoms or centers, which can thus give rise to optical isomers (enantiomers) and diastereomers. The present teachings include methods for preparing such optical isomers (enantiomers) and diastereomers (geometric isomers), as well as the racemic and resolved, enantiomerically pure (+) and (−) stereoisomers, as well as other mixtures of the (+) and (−) stereoisomers and pharmaceutically acceptable salts thereof. In some embodiments, optical isomers can be obtained in enantiomerically enriched or pure form by standard procedures known to those skilled in the art, which include, for example, chiral separation, diastereomeric salt formation, kinetic resolution, and asymmetric synthesis. The present teachings also encompass methods for preparing cis and trans isomers of compounds containing alkenyl moieties (e.g., alkenes and imines). It is also understood that the present teachings encompass all methods for making possible regioisomers in pure form and mixtures thereof, which can include standard separation procedures known to those skilled in the art, for examples, column chromatography, thin-layer chromatography, simulated moving-bed chromatography, and high-performance liquid chromatography.

One aspect of the present teachings provides a method for preparing a compound of formula VI or a tautomer thereof: 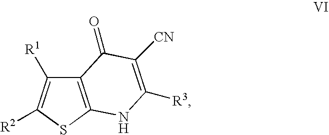
wherein:
R¹ is H, a halogen, a C₁₋₆ alkyl group, a C₆₋₁₄ aryl group, a 5-14 membered heteroaryl group, a —(C₁₋₆ alkyl)-C₆₋₁₄ aryl group, or a —(C₁₋₆ alkyl)-5-14 membered heteroaryl group, where each of the C₆₋₁₄ aryl groups and the 5-14 membered heteroaryl groups optionally is substituted with 1-4 groups independently selected from a halogen, a C₁₋₆ alkyl group, and a C₁₋₆ alkoxy group;
R² is H, a halogen, a C₁₋₆ alkyl group, a C₆₋₁₄ aryl group, a 5-14 membered heteroaryl group, a —(C₁₋₆ alkyl)-C₆₋₁₄ aryl group, or a —(C₁₋₆ alkyl)-5-14 membered heteroaryl group, where each of the C₆₋₁₄ aryl groups and the 5-14 membered heteroaryl groups optionally is substituted with 1-4 groups independently selected from a halogen, a C₁₋₆ alkyl group, and a C₁₋₆ alkoxy group; and R³ is H.

In some embodiments, R¹ can be H, a halogen, or a C₁₋₆ alkyl group. In certain embodiments, R¹ can be H. In certain embodiments, R¹ can be a halogen. For example, R¹ can be Br or I. In certain embodiments, R¹ can be a C₁₋₆ alkyl group. For example, R¹ can be a methyl group, an ethyl group, a propyl group, or a butyl group. In particular embodiments, R¹ can be a methyl group, an ethyl group, or an isopropyl group.

In some embodiments, R¹ can be a C₆₋₁₄ aryl group or a 5-14 membered heteroaryl group, where each of the C₆₋₁₄ aryl group and the 5-14 membered heteroaryl group can be optionally substituted with 1-4 groups independently selected from a halogen, a C₁₋₆ alkyl group, and a C₁₋₆ alkoxy group. In certain embodiments, R¹ can be a phenyl group optionally substituted with 1-4 groups independently selected from a halogen and a C₁₋₆ alkoxy group. For example, R¹ can be a phenyl group, a fluorophenyl group, a chlorophenyl group, a bromophenyl group, or a methoxyphenyl group. In particular embodiments, R¹ can be a phenyl group, a 4-fluorophenyl group, a 4-chlorophenyl group, a 4-bromophenyl group, or a 4-methoxyphenyl group. In certain embodiments, R¹ can be a 5-membered heteroaryl group. For example, R¹ can be a furanyl group.

In some embodiments, R¹ can be a —(C₁₋₆ alkyl)-C₆₋₁₄ aryl group or a —C₁₋₆ alkyl)₅₋₁₄-membered heteroaryl group, where each of the C₆₋₁₄ aryl group and the 5-14-membered heteroaryl group can be optionally substituted with 1-4 groups independently selected from a halogen, a C₁₋₆ alkyl group, and a C₁₋₆ alkoxy group. For example, R¹ can be a benzyl group.

In some embodiments, R² can be H, a halogen, or a C₁₋₆ alkyl group. In certain embodiments, R² can be H. In certain embodiments, R² can be a halogen. For example, R² can be Br or I. In certain embodiments, R² can be a C₁₋₆ alkyl group. For example, R² can be a methyl group, an ethyl group, a propyl group, an isopropyl group, or a butyl group. In particular embodiments, R² can be a methyl group or an ethyl group.

In some embodiments, R² can be a C₆₋₁₄ aryl group or a 5-14-membered heteroaryl group, where each of the C₆₋₁₄ aryl group and the 5-14 membered heteroaryl group can be optionally substituted with 1-4 groups independently selected from a halogen, a C₁₋₆ alkyl group, and a C₁₋₆ alkoxy group. In certain embodiments, R² can be a phenyl group optionally substituted with 1-4 groups independently selected from a halogen and a C₁₋₆ alkoxy group. For example, R² can be a phenyl group, a fluorophenyl group, a chlorophenyl group, a bromophenyl group, or a methoxyphenyl group. In particular embodiments, R² can be a phenyl group, a 4-fluorophenyl group, a 4-chlorophenyl group, a 4-bromophenyl group, or a 4-methoxyphenyl group. In certain embodiments, R² can be a 5-membered heteroaryl group. For example, R² can be a furanyl group.

In some embodiments, R² can be a —(C₁₋₆ alkyl)-C₆₋₁₄ aryl group or a —C₁₋₆ alkyl)-5-14-membered heteroaryl group, where each of the C₆₋₁₄ aryl group and the 5-14-membered heteroaryl group can be optionally substituted with 1-4 groups independently selected from a halogen, a C₁₋₆ alkyl group, and a C₁₋₆ alkoxy group. In particular embodiments, R² can be a benzyl group.

In some embodiments, the method can include heating a compound of formula IV: 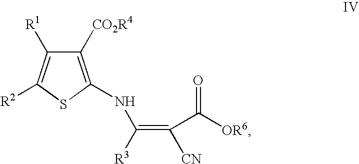
wherein R⁴ is a C₁₋₆ alkyl group, R⁶ is a group capable of forming a carbocation, and R¹, R², and R³ are as defined herein.

Without wishing to be bound to any particular theory, it is believed that upon heating, for example, under decarboxylation conditions, R⁶ of compound IV can undergo a thermal elimination with concomitant decarboxylation to give a cyanoacrylate of formula V as shown below. Accordingly, R⁶ can be any group capable of forming a carbocation. Groups that can form stabilized carbocations, e.g., tertiary carbocations, are expected to promote decarboxylation under these conditions. Thus, R⁶ groups can include a tertiary alkyl group such as a tert-butyl group, a 2-methylbut-2-yl group, and the like. R⁶ groups can also include groups that are not tertiary alkyl but can form tertiary or other stabilized carbocations, e.g., by proton or methyl migration, under the decarboxylation conditions. Such groups can include a neopentyl group, a 3-methylbut-2-yl group, and the like.

In some embodiments, the method can include heating the compound of formula IV in a solvent at a first elevated temperature. In certain embodiments, the method can include heating the solvent and adding the compound of formula IV to the heated solvent. The reaction mixture can be heated subsequently at a second elevated temperature that is the same as or different from (i.e., greater than or less than) the first elevated temperature.

In some embodiments, each of the first elevated temperature and the second elevated temperature can be between about 110° C. and about 300° C. In some embodiments, each of the first elevated temperature and the second elevated temperature can be between about 140° C. and about 300° C. In certain embodiments, each of the first elevated temperature and the second elevated temperature can be greater than 140° C. and less than 300° C. For example, each of the first elevated temperature and the second elevated temperature can be between about 140° C. and about 300° C., between about 150° C. and about 300° C., between about 160° C. and about 300° C., between about 170° C. and about 300° C., between about 180° C. and about 300° C., between about 190° C. and about 300° C., between about 200° C. and about 300° C., between about 220° C. and about 300° C., between about 240° C. and about 300° C., between about 260° C. and about 300° C., between about 150° C. and about 280° C., between about 160° C. and about 280° C., between about 170° C. and about 280° C., between about 180° C. and about 280° C., between about 190° C. and about 280° C., between about 200° C. and about 280° C., between about 210° C. and about 280° C., between about 230° C. and about 280° C., between about 150° C. and about 260° C., or between about 200° C. and about 260° C. In particular embodiments, each of the first elevated temperature and the second elevated temperature can be between about 200° C. and about 260° C., e.g., between about 250° and about 260° C.

In certain embodiments, the first elevated temperature can be between about 110° C. and about 260° C. In certain embodiments, the first elevated temperature can be greater than 110° C. and less than 260° C. For example, the first elevated temperature can be between about 120° C. and about 260° C., between about 130° C. and about 260° C., between about 140° C. and about 260° C., between about 150° C. and about 260° C., between about 160° C. and about 260° C., between about 170° C. and about 260° C., between about 180° C. and about 260° C., between about 190° C. and about 260° C., between about 200° C. and about 260° C., between about 210° C. and about 260° C., between about 220° C. and about 260° C., between about 230° C. and about 260° C., between about 120° C. and about 230° C., between about 130° C. and about 230° C., between about 140° C. and about 230° C., between about 150° C. and about 230° C., between about 160° C. and about 230° C., between about 170° C. and about 230° C., between about 180° C. and about 230° C., between about 190° C. and about 230° C., between about 200° C. and about 230° C., between about 210° C. and about 230° C., between about 120° C. and about 200° C., between about 130° C. and about 200° C., between about 140° C. and about 200° C., between about 150° C. and about 200° C., between about 160° C. and about 200° C., between about 170° C. and about 200° C., or between about 180° C. and about 200° C. In particular embodiments, the first elevated temperature can be about 200° C.

In some embodiments, the second elevated temperature can be different from (e.g., greater than) the first elevated temperature. In certain embodiments, the second elevated temperature can be between about 110° C. and about 300° C. (e.g., between about 140° C. and about 300° C.). For example, the second elevated temperature can be greater than 140° C. and less than 300° C. In particular embodiments, the second elevated temperature can be between about 250° and about 260° C. (e.g., about 256° C. or about 259° C.).

In some embodiments, the second elevated temperature can be the same as the first elevated temperature, for example, the method can include heating a compound of formula IV at a (single) elevated temperature to form the compound of formula VI or a tautomer thereof. In certain embodiments, the method can include heating the compound of formula IV in a solvent at the elevated temperature to provide the compound of formula VI. In particular embodiments, the method can include heating a solvent at the elevated temperature and adding the compound of formula IV into the heated solvent to provide a mixture. In particular embodiments, the method can further include heating the mixture at the elevated temperature to provide the compound of formula VI.

In some embodiments, the elevated temperature can be between about 140° C. and about 300° C. In certain embodiments, the elevated temperature can be greater than 140° C. and less than 300° C. For example, the elevated temperature can be between about 140° C. and about 300° C., between about 150° C. and about 300° C., between about 160° C. and about 300° C., between about 170° C. and about 300° C., between about 180° C. and about 300° C., between about 190° C. and about 300° C., between about 200° C. and about 300° C., between about 220° C. and about 300° C., between about 240° C. and about 300° C., between about 260° C. and about 300° C., between about 150° C. and about 280° C., between about 160° C. and about 280° C., between about 170° C. and about 280° C., between about 180° C. and about 280° C., between about 190° C. and about 280° C., between about 200° C. and about 280° C., between about 210° C. and about 280° C., between about 230° C. and about 280° C., between about 150° C. and about 260° C., or between about 200° C. and about 260° C. In particular embodiments, the elevated temperature can be between about 250° C. and about 260° C. (e.g., about 256° C. or about 259° C.).

In some embodiments, the solvent can have a boiling temperature of greater than or equal to 200° C. In certain embodiments, the solvent can have a boiling temperature between about 200° C. and about 300° C. In particular embodiments, the solvent can have a boiling temperature between about 250° C. and about 260° C. (e.g., about 256° C. or about 259° C.). In some embodiments, the solvent can include diphenyl ether, biphenyl, or a mixture thereof. In certain embodiments, the solvent can include diphenyl ether. In certain embodiments, the solvent can include biphenyl. In particular embodiments, the solvent can be selected from diphenyl ether, biphenyl, or a mixture thereof. In certain embodiments, the compound of formula IV can be dissolved in diphenyl ether or a solvent that includes diphenyl ether. In certain embodiments, the compound of formula IV can be dissolved in a mixture of biphenyl and diphenyl ether. In particular embodiments, the compound of formula IV can be dissolved in a eutectic mixture comprising about 26.5% of biphenyl and about 73.5% of diphenyl ether.

In some embodiments, the method can include providing the compound of formula IV in a solution having a concentration of less than or equal to 1 mole/liter (M). For example, the concentration can be less than or equal to 1M and greater than or equal to 0.1M. In certain embodiments, the concentration can be less than or equal to 0.5M and greater than or equal to 0.1M. In particular embodiments, the concentration can be about 0.2M.

In some embodiments, the method can include isolating the compound of formula V: 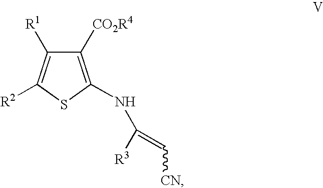
wherein R¹, R², R³, and R⁴ are as defined herein.

In some embodiments, the compound of formula IV can be prepared by treating a compound of formula III: 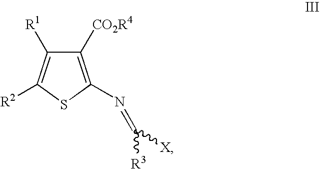
with an α-cyano ester (e.g., tert-butyl cyanoacetate), wherein X is —OR⁴ or —NR⁴R⁴, and R¹, R², R³, and R⁴ are as defined herein. In some embodiments, X can be —NR⁴R⁴. In some embodiments, the reaction of the compound of formula III with the α-cyano ester can be performed in tert-butanol or a solvent including tert-butanol. In certain embodiments, the reaction of compound III with the α-cyano ester can be performed at room temperature, for example, between about 20° C. and about 30° C.

In some embodiments, the compound of formula III can be prepared by treating a compound of formula I: 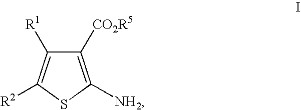
with a compound of formula II: 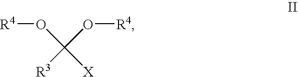
wherein R⁵ is H or a C₁₋₆ alkyl group, and R¹, R², R³, R⁴, and X are as defined herein.

Each instance of R⁴ can be the same or different. In certain embodiments, R⁵ can be a C₁₋₆ alkyl group. For example, R⁵ can be a methyl group, an ethyl group, a propyl group, an isopropyl group, a butyl group, or a t-butyl group. In some embodiments, the compound of formula I can be treated with triethyl orthoformate, trimethyl orthoacetate, dimethylformamide dimethyl acetal, or dimethylformamide diethyl acetal to provide the compound of formula III. In certain embodiments, the compound of formula I can be treated with dimethylformamide dimethyl acetal or dimethylformamide diethyl acetal to provide the compound of formula III. In some embodiments, compounds I and II can undergo a reaction to provide the compound of formula III in the absence of a solvent.

In some embodiments, the method can further include treating a compound of formula VI′: 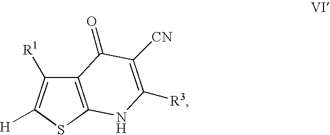
with an iodine source to form a compound of formula VI″: 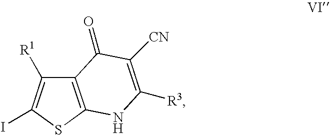
wherein R¹ and R³ are as defined herein. Examples of the iodine source include I₂ and ICI.

In some embodiments, the method can further include treating a compound of formula VI with a chlorinating reagent to provide a compound of formula VII: 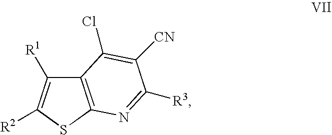
wherein R¹, R², and R³ are as defined herein.

In certain embodiments, the method can further include treating the compound of formula VI′ with a chlorinating reagent to form a compound of VII′: 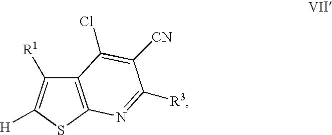
wherein R¹ and R³ are as defined herein.

In some embodiments, the method can further include treating the compound of formula VI″ with a chlorinating reagent to provide a compound of formula VII″: 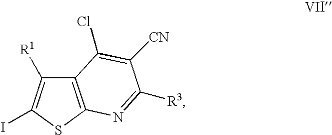
wherein R¹ and R³ are as defined herein. In the embodiments of preparing compounds of formula VII, formula VII′, or formula VII″, the chlorinating reagent can be selected from phosphorus oxychloride (POCl₃) and thionyl chloride (SOCl₂).

In some embodiments, the method can further include converting a compound of formula VII, where R¹ is H, into a compound of formula VIII: 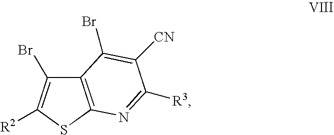
wherein R² and R³ are as defined herein. In particular embodiments, the compound of formula VIII can be prepared by treating the compound of formula VII, where R¹ is H, with a brominating agent, for example, bromine.

Another aspect of the present teachings provides a method for preparing a compound of formula VII″ or a tautomer thereof, and converting it into a compound described in U.S. Patent Application Publication No. 2007/0082880 A1 (“the '880 publication”). In some embodiments, the method can include converting the compound of formula VII″ into a compound of formula XI: 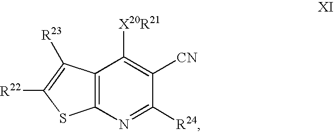
wherein:
X²⁰ is a) —NR²⁵—Y²⁰—, b) —O—Y²⁰—, c) —S(O)ₘ—Y²⁰—, d) —S(O)ₘNR²⁵—Y²⁰—, e) —NR²⁵S(O)ₘ—Y²⁰—, f) —C(O)NR²⁵—Y²⁰—, g) —NR²⁵C(O)—Y²⁰—, h) —C(S)NR²⁵—Y²⁰—, i) —NR²⁵C(S)—Y²⁰—, j) —C(O)O—Y²⁰—, k) —OC(O)—Y²⁰—, l) —C(O)—Y²⁰—, or m) a covalent bond;
Y²⁰, at each occurrence, is a) a divalent C₁₋₁₀ alkyl group, b) a divalent C₂₋₁₀ alkenyl group, c) a divalent C₂₋₁₀ alkynyl group, d) a divalent C₁₋₁₀ haloalkyl group, or e) a covalent bond;
R²¹ is a) a C₁₋₁₀ alkyl group, b) a C₃₋₁₀ cycloalkyl group, c) a 3-12 membered cycloheteroalkyl group, d) a C₆₋₁₄ aryl group, or e) a 5-13 membered heteroaryl group, wherein each of a)-e) optionally is substituted with 1-4 R²⁶;
R²² is a) H, b) halogen, c) —C(O)R²⁸, d) —C(O)OR²⁸, e) —C(O)NR²⁹R³⁰, f) —C(S)R²⁸, g) —C(S)OR²⁸, h) —C(S)NR²⁹R³⁰, i) a C₁₋₁₀ alkyl group, j) a C₂₋₁₀ alkenyl group, k) a C₂₋₁₀ alkynyl group, l) a C₃₋₁₀ cycloalkyl group, m) a C₆₋₁₄ aryl group, n) a 3-12 membered cycloheteroalkyl group, or o) a 5-13 membered heteroaryl group, wherein each of i)-o) optionally is substituted with 1-4 R²⁶ groups;
R²³ is a) H, b) halogen, c) —OR²⁸, d) —NR²⁹R³⁰, e) —N(O)R²⁹R³⁰, f) —S(O)ₘR²⁸, g) —S(O)ₘOR²⁸, h) —C(O)R²⁸, i) —C(O)OR²⁸, j) —C(O)NR²⁹R³⁰, k) —C(S)R²⁸, l) —C(S)OR²⁸, m) —C(S)NR²⁹R³⁰, n) —Si(C₁₋₁₀ alkyl group)₃, o) a C₁₋₁₀ alkyl group, p) a C₂₋₁₀ alkenyl group, q) a C₂₋₁₀ alkynyl group, r) a C₃₋₁₀ cycloalkyl group, s) a C₆₋₁₄ aryl group, t) a 3-12 membered cycloheteroalkyl group, or u) a 5-13 membered heteroaryl group, wherein each of o)-u) optionally is substituted with 1-4 R²⁶ groups;
R²⁴ is a) H, b) halogen, c) a C₁₋₁₀ alkyl group, d) a C₂₋₁₀ alkenyl group, e) a C₂₋₁₀ alkynyl group, f) a C₁₋₁₀ haloalkyl group, g) a C₃₋₁₀ cycloalkyl group, h) a C₆₋₁₄ aryl group, i) a 3-12 membered cycloheteroalkyl group, or j) a 5-13 membered heteroaryl group, wherein each of c)-j) optionally is substituted with 1-4 R²⁶ groups;
R²⁵, at each occurrence, is a) H, b) a C₁₋₁₀ alkyl group, c) a C₂₋₁₀ alkenyl group, d) a C₂₋₁₀ alkynyl group, or e) a C₁₋₁₀ haloalkyl group;
R²⁶, at each occurrence, is a) R²⁷ or b) —Y²⁰—R²⁷;
R²⁷, at each occurrence, is a) halogen, b) —CN, c) —NO₂, d) oxo, e) —OR²⁸, f) —NR²⁹R³⁰ g) —N(O)R²⁹R³⁰, h) —S(O)ₘR²⁸, i) —S(O)ₘOR²⁸, j) —SO₂NR²⁹R³⁰, k) —C(O)R²⁸, l) —C(O)OR²⁸, m) —C(O)NR²⁹R³⁰, n) —C(S)R²⁸, o) —C(S)OR²⁸, p) —C(S)NR²⁹R³⁰, q) —Si(C₁₋₁₀ alkyl)₃, r) a C₁₋₁₀ alkyl group, s) a C₂₋₁₀ alkenyl group, t) a C₂₋₁₀ alkynyl group, u) a C₁₋₁₀ haloalkyl group, v) a C₃₋₁₀ cycloalkyl group, w) a C₆₋₁₄ aryl group, x) a 3-12 membered cycloheteroalkyl group, or y) a 5-13 membered heteroaryl group, wherein each of r)-y) optionally is substituted with 1-4 R³¹ groups;
R²⁸, at each occurrence, is a) H, b) —C(O)R³⁴, c) —C(O)OR³⁴, d) a C₁₋₁₀ alkyl group, e) a C₂₋₁₀ alkenyl group, f) a C₂₋₁₀ alkynyl group, g) a C₁₋₁₀ haloalkyl group, h) a C₃₋₁₀ cycloalkyl group, i) a C₆₋₁₄ aryl group, j) a 3-12 membered cycloheteroalkyl group, or k) a 5-13 membered heteroaryl group, wherein each of d)-k) optionally is substituted with 1-4 R³¹ groups;
R²⁹ and R³⁰, at each occurrence, independently are a) H, b) —OR³³, c) —NR³⁴R³⁵, d) —S(O)ₘR³⁴, e) —S(O)ₘOR³⁴, f) —S(O)₂NR³⁴R³⁵, g) —C(O)R³⁴, h) —C(O)OR³⁴, i) —C(O)NR³⁴R³⁵, j) —C(S)R³⁴, k) —C(S)OR³⁴, l) —C(S)NR³⁴R³⁵, m) a C₁₋₁₀ alkyl group, n) a C₂₋₁₀ alkenyl group, o) a C₂₋₁₀ alkynyl group, p) a C₁₋₁₀ haloalkyl group, q) a C₃₋₁₀ cycloalkyl group, r) a C₆₋₁₄ aryl group, s) a 3-12 membered cycloheteroalkyl group, or t) a 5-13 membered heteroaryl group, wherein each of m)-t) optionally is substituted with 1-4 R³¹ groups;
R³¹, at each occurrence, is a) R³² or b) —Y²⁰—R³²;
R³², at each occurrence, is a) halogen, b) —CN, c) —NO₂, d) oxo, e) —OR³³, f) —NR³⁴R³⁵, g) —N(O)R³⁴R³⁵ h) —S(O)ₘR³³, i) —S(O)ₘOR³³, j) —SO₂NR³⁴R³⁵, k) —C(O)R³³, l) —C(O)OR³³, m) —C(O)NR³⁴R³⁵, n) —C(S)R³³, o) —C(S)OR³³, p) —C(S)NR³⁴R³⁵, q) —Si(C₁₋₁₀ alkyl)₃, r) a C₁₋₁₀ alkyl group, s) a C₂₋₁₀ alkenyl group, t) a C₂₋₁₀ alkynyl group, u) a C₁₋₁₀ haloalkyl group, v) a C₃₋₁₀ cycloalkyl group, w) a C₆₋₁₄ aryl group, x) a 3-12 membered cycloheteroalkyl group, or y) a 5-13 membered heteroaryl group, wherein each of r)-y) optionally is substituted with 1-4 R³⁶ groups;
R³³, at each occurrence, is selected from a) H, b) —C(O)R³⁴, c) —C(O)OR³⁴, d) a C₁₋₁₀ alkyl group, e) a C₂₋₁₀ alkenyl group, f) a C₂₋₁₀ alkynyl group, g) a C₁₋₁₀ haloalkyl group, h) a C₃₋₁₀ cycloalkyl group, i) a C₆₋₁₄ aryl group, j) a 3-12 membered cycloheteroalkyl group, and k) a 5-13 membered heteroaryl group, wherein each of d)-k) optionally is substituted with 1-4 R³⁶ groups;
R³⁴ and R³⁵, at each occurrence, independently are a) H, b) a C₁₋₁₀ alkyl group, c) a C₂₋₁₀ alkenyl group, d) a C₂₋₁₀ alkynyl group, e) a C₁₋₁₀ haloalkyl group, f) a C₃₋₁₀ cycloalkyl group, g) a C₆₋₁₄ aryl group, h) a 3-12 membered cycloheteroalkyl group, or i) a 5-13 membered heteroaryl group, wherein each of b)-i) optionally is substituted with 1-4 R³⁶ groups;
R³⁶, at each occurrence, is a) halogen, b) —CN, c) —NO₂, d) —OH, e) —NH₂, f) —NH(C₁₋₁₀ alkyl), g) oxo, h) —N(C₁₋₁₀ alkyl)₂, i) —SH, j) —S(O)ₘ—C₁₋₁₀ alkyl, k) —S(O)₂OH, l) —S(O)ₘ—OC₁₋₁₀ alkyl, m) —C(O)—C₁₋₁₀ alkyl, n) —C(O)OH, o) —C(O)—OC₁₋₁₀ alkyl, p) —C(O)NH₂, q) —C(O)NH—C₁₋₁₀ alkyl, r) —(O)N(C₁₋₁₀ alkyl)₂, s) —C(S)NH₂, t) —C(S)NH—C₁₋₁₀ alkyl, u) —C(S)N(C₁₋₁₀ alkyl)₂, v) a C₁₋₁₀ alkyl group, w) a C₂₋₁₀ alkenyl group, x) a C₂₋₁₀ alkynyl group, y) a C₁₋₁₀ alkoxy group, z) a C₁₋₁₀ haloalkyl group, aa) a C₃₋₁₀ cycloalkyl group, ab) a C₆₋₁₄ aryl group, ac) a 3-12 membered cycloheteroalkyl group, or ad) a 5-13 membered heteroaryl group; and
m is 0, 1, or 2;
or a pharmaceutically acceptable salt thereof.

In some embodiments, the method can include converting the compound of formula VII″ into a compound of formula XI′: 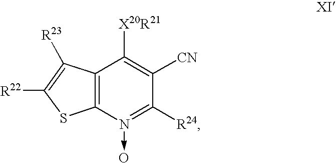
wherein R²¹-R²⁴ and X²⁰ are defined herein.

In some embodiments, the method can include converting the compound of formula VII″ into a compound of formula XI″: 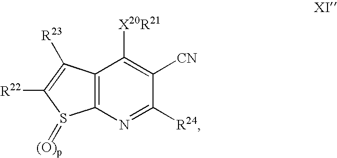
wherein p is 1 or 2, and R²¹-R²⁴ and X²⁰ are defined herein.

In some embodiments, X²⁰ can be —NR²⁵—Y²⁰—, —O—, —NR²⁵C(O)—, or a covalent bond. For example, R²⁵ can be H or a C₁₋₆ alkyl group and Y²⁰ can be a covalent bond or a divalent C₁₋₆ alkyl group. In certain embodiments, X²⁰ can be —NH—, —N(CH₃)—, —NH—CH₂—, —NH—(CH₂)₂—, —N(CH₃)CH₂—, —O—, —NHC(O)—, —N(CH₃)C(O)—, or a covalent bond.

In some embodiments, R²¹ can be a 5-13 membered heteroaryl group optionally substituted with 1-4 R²⁶ groups. For example, R²¹ can be an indolyl group, a benzimidazolyl group, a pyrrolo[2,3-b]pyridinyl group, a pyridinyl group, or an imidazolyl group, each of which can be optionally substituted with 1-4 R²⁶ groups.

In certain embodiments, R²¹ can be an indolyl group optionally substituted with 1-4 R²⁶ groups and can be connected to X²⁰ or the thienopyridine ring at any of the available carbon ring atoms. For example, R²¹ can be a 1H-indol-5-yl group, a 1H-indol-4-yl group, a 1H-indol-7-yl group, a 1H-indol-6-yl group, a 4-methyl-1H-indol-5-yl group, a 2-methyl-1H-indol-5-yl group, a 7-methyl-1H-indol-5-yl group, a 3-methyl-1H-indol-5-yl group, a 1-methyl-1H-indol-5-yl group, a 6-methyl-1H-indol-5-yl group, or a 4-ethyl-1H-indol-5-yl group.

In certain embodiments, R²¹ can be a 1H-benzimidazol-5-yl group, a 1H-benzimidazol-4-yl group, a 1H-pyrrolo[2,3-b]pyridin-5-yl group, a 1H-pyrrolo[2,3-b]pyridin-4-yl group, a pyridin-3-yl group, or a pyridin-4-yl group, each of which can be optionally substituted with 1-4 R²⁶ groups. For example, R²¹ can be a 4-chloro-1H-pyrrolo[2,3-b]pyridin-5-yl group or a 4-chloro-1-[(4-methylphenyl)sulfonyl]-1H-pyrrolo[2,3-b]pyridin-5-yl group.

In some embodiments, R²² can be H, a halogen, —C(O)R²⁸, —C(O)OR²⁸, or —C(O)NR²⁹R³⁰. In certain embodiments, R²² can be H, Cl, Br, I, —C(O)R²⁸, —C(O)OR²⁸, or —C(O)NR²⁹R³⁰. For example, R²⁸, R²⁹, and R³⁰ can independently be H, a C₁₋₁₀ alkyl group, a 3-12 membered cycloheteroalkyl group, a 5-13 membered heteroaryl group, or a phenyl group, where each of the C₁₋₁₀ alkyl group, the 3-12 membered cycloheteroalkyl group, the 5-13 membered heteroaryl group, and the phenyl group can be optionally substituted with 1-4 R³¹ groups.

In some embodiments, R²² can be a C₁₋₁₀ alkyl group, a C₂₋₁₀ alkenyl group, a C₂₋₁₀ alkynyl group, a C₃₋₁₀ cycloalkyl group, a 3-12 membered cycloheteroalkyl group, a C₆₋₁₄ aryl group, or a 5-13 membered heteroaryl group, each of which can be optionally substituted with 1-4 R²⁶ groups. For example, R²⁶ can be a halogen, oxo, —OR²⁸, —NR²⁹R³⁰, —S(O)₂R²⁸, —S(O)₂OR²⁸, —SO₂NR²⁹R³⁰, —C(O)R²⁸, —C(O)OR²⁸, —C(O)NR²⁹R³⁰, —Si(CH₃)₃, —C₁₋₄ alkyl)-OR²⁸, —C₁₋₄ alkyl-NR²⁹R³⁰, a —C₁₋₄ alkyl-C₆₋₁₄ aryl group, a —C₁₋₄ alkyl-3-12 membered cycloheteroalkyl group, a —C₁₋₄ alkyl-5-13 membered heteroaryl group, a C₁₋₁₀ alkyl group, a C₂₋₁₀ alkenyl group, a C₂₋₁₀ alkynyl group, a C₁₋₁₀ haloalkyl group, a C₃₋₁₀ cycloalkyl group, a C₆₋₁₄ aryl group, a 3-12 membered cycloheteroalkyl group, or a 5-13 membered heteroaryl group, where each of the C₁₋₁₀ alkyl groups, the C₂₋₁₀ alkenyl group, the C₂₋₁₀ alkynyl group, the C₃₋₁₀ cycloalkyl group, the C₆₋₁₄ aryl groups, the 3-12 membered cycloheteroalkyl groups, and the 5-13 membered heteroaryl groups can be optionally substituted with 1-4 R³¹ groups.

In certain embodiments, R²² can be a C₁₋₆ alkyl group, a C₂₋₆ alkenyl group, or a C₂₋₆ alkynyl group, each of which can be optionally substituted 1-4 R²⁶ groups, where R²⁶, at each occurrence, can be a halogen, —OR²⁸, —NR²⁹R³⁰, —C(O)R²⁸, —C(O)OR²⁸, —C(O)NR²⁹R³⁰, —Si(CH₃)₃, a phenyl group, a 5-6 membered cycloheteroalkyl group, or a 5-6 membered heteroaryl group, and each of the phenyl group, the 5-6 membered cycloheteroalkyl group, and the 5-6 membered heteroaryl group can be optionally substituted with 1-4 R³¹ groups.

In the embodiments where R²² can be a C₁₋₆ alkyl group, a C₂₋₆ alkenyl group, or a C₂₋₆ alkynyl group, R²⁸, at each occurrence, can be H, a C₁₋₆ alkyl group, a phenyl group, a 5-6 membered cycloheteroalkyl group, or a 5-6 membered heteroaryl group, where each of the C₁₋₆ alkyl groups, the phenyl group, the 5-6 membered cycloheteroalkyl group, and the 5-6 membered heteroaryl group can be optionally substituted with 1-4 R³¹ groups; and R²⁹ and R³⁰, at each occurrence, independently can be H, —N(C₁₋₆ alkyl)₂, a C₁₋₆ alkyl group, a phenyl group, a 5-6 membered cycloheteroalkyl group, or a 5-6 membered heteroaryl group, where each of the C₁₋₆ alkyl group, the phenyl group, the 5-6 membered cycloheteroalkyl group, and the 5-6 membered heteroaryl group can be optionally substituted with 1-4 R³¹ groups. In certain embodiments, each of R²⁸, R²⁹, and R³⁰ can be a piperazinyl group, a piperidinyl group, a pyrrolidinyl group, a morpholinyl group, a pyrazolyl group, a pyrimidinyl group, or a pyridinyl group, each of which can be optionally substituted with 1-4 R³¹ groups, where R³¹, at each occurrence, can be a halogen, —OR³³, —NR³⁴R³⁵, —C(O)NR³⁴R³⁵, a C₁₋₆ alkyl group, a C₁₋₆ alkoxy group, a C₁₋₆ haloalkyl group, —C₁₋₄ alkyl-NR³⁴R³⁵, a —C₁₋₄ alkyl-phenyl group, a —C₁₋₄ alkyl-5-6 membered cycloheteroalkyl group, or a —C₁₋₄ alkyl-5-6 membered heteroaryl group.

In certain embodiments, R²² can be a C₃₋₆ cycloalkyl group, a 3-10 membered cycloheteroalkyl group, a C₆₋₁₀ aryl group, or a 5-10 membered heteroaryl group, each of which can be optionally substituted with 1-4 R²⁶ groups. For example, R²² can be a cyclohexyl group, a cyclohexenyl group, a piperazinyl group, a piperidinyl group, a morpholinyl group, a pyrrolidinyl group, a tetrahydropyridinyl group, a dihydropyridinyl group, a phenyl group, a naphthyl group, a pyridinyl group, a pyrazolyl group, a pyridazinyl group, an indolyl group, a pyrazinyl group, a pyrimidinyl group, a thienyl group, a furyl group, a thiazolyl group, a quinolinyl group, a benzothienyl group, or an imidazolyl group, each of which can be optionally substituted with 1-4 R²⁶ groups.

In the embodiments where R²² can be a C₃₋₆ cycloalkyl group, a 3-10 membered cycloheteroalkyl group, a C₆₋₁₀ aryl group, or a 5-10 membered heteroaryl group, R²⁶, at each occurrence, can be a halogen, oxo, —OR²⁸, —NR²⁹R³⁰, —S(O)₂R²⁸, —S(O)₂OR²⁸, —SO₂NR²⁹R³⁰, —C(O)R²⁸, —C(O)OR²⁸, —C(O)NR²⁹R³⁰, a C₁₋₁₀ alkyl group, a C₃₋₁₀ cycloalkyl group, a C₆₋₁₄ aryl group, a 3-12 membered cycloheteroalkyl group, or a 5-13 membered heteroaryl group, where each of the C₁₋₁₀ alkyl group, the C₃₋₁₀ cycloalkyl group, the C₆₋₁₄ aryl group, the 3-12 membered cycloheteroalkyl group, and the 5-13 membered heteroaryl group can be optionally substituted with 1-4 R³¹ groups.

In particular embodiments, R²² can be a phenyl group optionally substituted with 1-4 R²⁶ groups independently selected from a halogen, —OR²⁸, —NR²⁹R³⁰, —S(O)₂R²⁸, —SO₂NR²⁹R³⁰, —C(O)R²⁸, —C(O)OR²⁸, —C(O)NR²⁹R³⁰, a C₁₋₆ alkyl group, a C₃₋₆ cycloalkyl group, a C₆₋₁₀ aryl group, a 3-10 membered cycloheteroalkyl group, and a 5-10 membered heteroaryl group, where each of the C₁₋₆ alkyl group, the C₃₋₆ cycloalkyl group, the C₆₋₁₀ aryl group, the 3-10 membered cycloheteroalkyl group, and the 5-10 membered heteroaryl group can be optionally substituted with 1-4 R³¹ groups. For example, R²² can be a phenyl group optionally substituted with 1-4 groups independently selected from a cyclohexyl group, a cyclohexenyl group, a piperazinyl group, a piperidinyl group, a morpholinyl group, a pyrrolidinyl group, a tetrahydropyridinyl group, a dihydropyridinyl group, a phenyl group, a naphthyl group, a pyridinyl group, a pyrazolyl group, a pyridazinyl group, an indolyl group, a pyrazinyl group, a pyrimidinyl group, a thienyl group, a furyl group, a thiazolyl group, a quinolinyl group, a benzothienyl group, and an imidazolyl group, each of which can be optionally substituted with 1-4 R³¹ groups.

In the embodiments where R²² can be a C₃₋₆ cycloalkyl group, a 3-10 membered cycloheteroalkyl group, a C₆₋₁₀ aryl group, or a 5-10 membered heteroaryl group, R²⁸, at each occurrence, can be H, a C₁₋₆ alkyl group, a phenyl group, a 5-6 membered cycloheteroalkyl group, or a 5-6 membered heteroaryl group, where each of the C₁₋₆ alkyl group, the phenyl group, the 5-6 membered cycloheteroalkyl group, and the 5-6 membered heteroaryl group can be optionally substituted with 1-4 R³¹ groups; and R²⁹ and R³⁰, at each occurrence, independently can be H, —C(O)NR³⁴R³⁵, —S(O)₂R³⁴, —S(O)₂NR³⁴R³⁵, —NR³⁴R³⁵, a C₁₋₆ alkyl group, a phenyl group, a 5-6 membered cycloheteroalkyl group, or a 5-6 membered heteroaryl group, where each of the C₁₋₆ alkyl group, the phenyl group, the 5-6 membered cycloheteroalkyl group, and the 5-6 membered heteroaryl group can be optionally substituted with 1-4 R³¹ groups. For example, each of R²⁸, R²⁹, and R³⁰ can be a piperazinyl group, a piperidinyl group, a pyrrolidinyl group, a morpholinyl group, a pyrazolyl group, a pyrimidinyl group, or a pyridinyl group, each of which can be optionally substituted with 1-4 R³¹ groups, where R³¹, at each occurrence, can be a halogen, —OR³³, —NR³⁴R³⁵, —C(O)NR³⁴R³⁵, a C₁₋₆ alkyl group, a C₁₋₆ alkoxy group, a C₁₋₆ haloalkyl group, —C₁₋₂ alkyl-NR³⁴R³⁵, a —C₁₋₂ alkyl-phenyl group, a —C 2 alkyl-5-6 membered cycloheteroalkyl group, or a —C₁₋₂ alkyl-5-6 membered heteroaryl group.

In certain embodiments, R²² can have the formula -A²⁰-J²⁰-G²⁰, where A²⁰ can be a divalent C₂₋₁₀ alkenyl group, a divalent C₂₋₁₀ alkynyl group, a divalent C₃₋₁₀ cycloalkyl group, a divalent 3-12 membered cycloheteroalkyl group, a divalent C₆₋₁₄ aryl group, or a divalent 5-13 membered heteroaryl group, J²⁰ can be a divalent C₁₋₁₀ alkyl group or a covalent bond, and G²⁰ can be selected from H, —S(O)ₘR²⁸, —S(O)ₘOR²⁸, —SO₂NR²⁹R³⁰, —C(O)R²⁸, —C(O)OR²⁸, —C(O)NR²⁹R³⁰, —NR²⁹R³⁰, a 3-12 membered cycloheteroalkyl group, a C₆₋₁₄ aryl group, and a 5-13 membered heteroaryl group, where each of the 3-12 membered cycloheteroalkyl group, the C₆₋₁₄ aryl group, and the 5-13 membered heteroaryl group can be optionally substituted with 1-4 R³¹ groups. In some embodiment, A²⁰ can be optionally substituted with 1-3 R²⁶ groups in addition to the -J²⁰-G²⁰ group.

In some embodiments, A²⁰ can be a phenyl group, J²⁰ can be a divalent-C₁₋₂ alkyl group, and G²⁰ can be a 3-12 membered cycloheteroalkyl group optionally substituted with 1-4 R³¹ groups. Examples of G²⁰ can include, but are not limited to, a pyrrolidinyl group, a piperidinyl group, a piperazinyl group, and a morpholinyl group. In certain embodiments, G²⁰ can be an N-substituted piperazinyl group and the substitution group can have the formula —(CH₂)ₙ-D²⁰, where n can be 1, 2, or 3, and D²⁰ can be selected from H, —OR³³, —NR³⁴R³⁵, —C(O)R³³, a 3-12 membered cycloheteroalkyl group, a C₆₋₁₄ aryl group, and a 5-13 membered heteroaryl group.

In some embodiments, G²⁰ can be —NR²⁹R³⁰, where R²⁹ can be H or a C₁₋₁₀ alkyl group optionally substituted with 1-4 —OR³¹, and R³⁰ can be H or a C₁₋₁₀ alkyl group optionally substituted with 1-4 substituents independently selected from —OR³³, —NR³⁴R³⁵, and a 3-10 membered cycloheteroalkyl group.

In some embodiments, A²⁰ can be selected from a divalent thienyl group, a divalent furanyl group, a divalent imidazolyl group, a divalent 1-methyl-imidazolyl group, a divalent thiazolyl group, and a divalent pyridinyl group.

In some embodiments, A²⁰ can be a divalent C₂₋₁₀ alkenyl group or a divalent C₂₋₁₀ alkynyl group, J²⁰ can be a covalent bond, and G²⁰ can be selected from —NR²⁹R³⁰, —Si(C₁₋₆ alkyl)₃, a 3-12 membered cycloheteroalkyl group, a C₆₋₁₄ aryl group, and a 5-13 membered heteroaryl group, where each of the 3-12 membered cycloheteroalkyl group, the C₆₋₁₄ aryl group, and the 5-13 membered heteroaryl group can be optionally substituted with 1-4 R³¹ groups. For example, R³¹ can be selected from —NR³⁴R³⁵, —C₁₋₂ alkyl-NR³⁴R³⁵, and a —C₁₋₂ alkyl-3-12 membered cycloheteroalkyl group, where the 3-12 membered cycloheteroalkyl group can be optionally substituted with 1-4 R³⁶ groups.

In some embodiments, R²³ can be H, a halogen, a C₁₋₆ alkyl group, a C₂₋₆ alkynyl group, or a phenyl group, where each of the C₁₋₆ alkyl group, the C₂₋₆ alkynyl group, and the phenyl group can be optionally substituted with 1-4 R²⁶ groups. For example, R²⁶, at each occurrence, can be —NR²⁹R³⁰, a C₁₋₆ alkyl group, a phenyl group, or a 5-10 cycloheteroalkyl group, where each of the C₁₋₆ alkyl group, the phenyl group, and the 5-10 cycloheteroalkyl group can be optionally substituted with 1-4 R³¹ groups.

In some embodiments, R²⁴ can be H.

Another aspect of the present teachings provides a method of preparing a compound of formula VII″ or a tautomer thereof, and converting it into a compound described in U.S. Pat. No. 6,987,116 B2 (“the '116 patent”). In some embodiments, the method can include converting the compound of formula VII″ into a compound of formula XII: 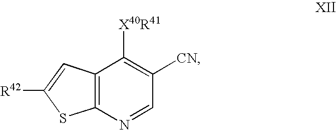
wherein:
X⁴⁰ is —NH—, —NR⁴⁴—, —O—, —S(O)ₘ—, or —NHCH₂—;
m is 0, 1, or 2;
n is 2, 3, 4, or 5;
q is 0, 1, 2, 3, 4, or 5;
R⁴¹ is a phenyl ring optionally substituted with one to four substituents independently selected from -J, —NO₂, —CN, —N₃, —CHO, —CF₃, —OCF₃, —R⁴⁴, —OR⁴⁴, —S(O)ₘR⁴⁴, —NR⁴⁴R⁴⁴, —NR⁴⁴S(O)ₘR⁴⁴, —OR⁴⁶OR⁴⁴, —OR⁴⁶NR⁴⁴R⁴⁴, —N(R⁴⁴)R⁴⁶OR⁴⁴, —N(R⁴⁴)R⁴⁶NR⁴⁴R⁴⁴, —NR⁴⁴C(O)R⁴⁴, —C(O)R⁴⁴, —C(O)OR⁴⁴, —C(O)NR⁴⁴R⁴⁴, —OC(O)R⁴⁴, —OC(O)OR⁴⁴, —OC(O)NR⁴⁴R⁴⁴, —NR⁴⁴C(O)R⁴⁴, —NR⁴⁴C(O)OR⁴⁴, —NR⁴⁴C(O)NR⁴⁴R⁴⁴, —R⁴⁵OR⁴⁴, —R⁴⁵NR⁴⁴R⁴⁴, —R⁴⁵S(O)ₘR⁴⁴, —R⁴⁵—C(O)R⁴⁴, —R⁴⁵C(O)OR⁴⁴, —R⁴⁵C(O)NR⁴⁴R⁴⁴, —R⁴⁵OC(O)R⁴⁴, —R⁴⁵OC(O)OR⁴⁴, —R⁴⁵OC(O)NR⁴⁴R⁴⁴, —R⁴⁵NR⁴⁴C(O)R⁴⁴, —R⁴⁵NR⁴⁴C(O)OR⁴⁴, —R⁴⁵NR⁴⁴C(O)NR⁴⁴R⁴⁴, and —Y⁴⁰R⁴⁷;
R⁴² is —H, —R⁴³, -J, —C(O)X⁴⁰R⁴³, or —CHO;
R⁴³ is a C₁₋₆ alkyl group, a C₂₋₆ cis-alkenyl group, a C₂₋₆ trans-alkenyl group, a C₂₋₆ alkynyl group, a C₆₋₁₄ aryl group, or a 5-14 membered heteroaryl group, each of which optionally is substituted by one or more groups selected from —C(O)X⁴⁰R⁴⁸, —CHO, —C(O)Q, 1,3-dioxolane, —R⁴⁸, —(C(R⁴⁹)₂)_{q}X⁴⁰R⁴⁸, —(C(R⁴⁹)₂)_{q}Q, —X⁴⁰(C(R⁴⁹)₂)ₙX⁴⁰R⁴⁸, —X⁴⁰(C(R⁴⁹)₂)ₙQ, and —X⁴⁰(C(R⁴⁹)₂)_{q}R⁴⁸;
R⁴⁴ is H, a C₁₋₆ alkyl group, a C₂₋₆ cis-alkenyl group, a C₂₋₆ trans-alkenyl group, or a C₂₋₆ alkynyl group;
R⁴⁵ is a divalent group selected from a C₁₋₆ alkyl group, a C₂₋₆ alkenyl group, and a C₂₋₆ alkynyl group;
R⁴⁶ is a divalent C₂₋₆ alkyl group;
R⁴⁷ is a C₃₋₇ cycloalkyl group, a C₆₋₁₄ aryl group, or a 5-14 membered heteroaryl group, a C₆₋₁₄ aryl or a 5-14 membered heteroaryl fused to one to three C₆₋₁₄ aryl or 5-14 membered heteroaryl groups, wherein each of the aryl groups, the cycloalkyl group, or the heteroaryl groups optionally is substituted with one to four substituents independently selected from a C₆₋₁₄ aryl group, —CH₂—C₆₋₁₄ aryl group, —NH—C₆₋₁₄ aryl group, —O—C₆₋₁₄ aryl group, —S(O)ₘ—C₆₋₁₄ aryl group, -J, —NO₂, —CN, —N₃, —CHO, —CF₃, —OCF₃, —R⁴⁴, —OR⁴⁴, —S(O)ₘR⁴⁴—NR⁴⁴R⁴⁴, —NR⁴⁴S(O)ₘR⁴⁴, —OR⁴⁶OR⁴⁴, —OR⁴⁶NR⁴⁴R⁴⁴, —N(R⁴⁴)R⁴⁶OR⁴⁴, —N(R⁴⁴)R⁴⁶NR⁴⁴R⁴⁴, —NR⁴⁴C(O)⁴⁴, —C(O)R⁴⁴, —C(O)OR⁴⁴, —C(O)NR⁴⁴R⁴⁴, —OC(O)R⁴⁴, —OC(O)OR⁴⁴, —OC(O)NR⁴⁴R⁴⁴, —NR⁴⁴C(O)R⁴⁴, —NR⁴⁴C(O)OR⁴⁴, —NR⁴⁴C(O)NR⁴⁴R⁴⁴, —R⁴⁵OR⁴⁴, —R⁴⁵NR⁴⁴R⁴⁴, —R⁴⁵S(O)ₘR⁴⁴, —R⁴⁵—C(O)R⁴⁴, —R⁴⁵C(O)OR⁴⁴, —R⁴⁵C(O)NR⁴⁴R⁴⁴, —R⁴⁵C(O)R⁴⁴, —R⁴⁵C(O)OR⁴⁴, —R⁴⁵C(O)NR⁴⁴R⁴⁴, —R⁴⁵OC(O)R⁴⁴, —R⁴⁵OC(O)OR⁴⁴, —R⁴⁵OC(O)NR⁴⁴R⁴⁴, —R⁴⁵NR⁴⁴C(O)R⁴⁴, —R⁴⁵NR⁴⁴C(O)OR⁴⁴, and —R⁴⁵NR⁴⁴C(O)NR⁴⁴R⁴⁴;
R⁴⁸ is H, a C₁₋₆ alkyl group, a C₂₋₆ cis-alkenyl group, a C₂₋₆ trans-alkenyl group, a C₂₋₆ alkynyl group, a C₆₋₁₄ aryl group, or a 5-14 membered heteroaryl group;
R⁴⁹ is —R⁴⁴ or —F;
Y⁴⁰ is —C(O)—, —C(O)O—, —OC(O)—, —C(O)NH—, —NHC(O), —NHSO₂—, —SO₂NH—, —C(OH)H—, —X⁴⁰(C(R⁴⁹)₂)_{q}—, —C(R⁴⁹)₂)_{q}—, —C(R⁴⁹)₂)_{q}X⁴⁰—, —C≡C—, cis- or trans-—CH═CH—, or a divalent C₃₋₁₀ cycloalkyl group;
Q is NZZ′ wherein Z and Z′ are the same or different and are independently H, a C₁₋₆ alkyl group, a C₂₋₆ alkenyl group, a C₂₋₆ alkynyl group, a C₆₋₁₄ aryl group, or a 5-14 membered heteroaryl group;
Z and Z′ taken together with the nitrogen to which they are attached form a 3-14 membered heterocyclic ring which optionally has an additional heteroatom selected from nitrogen, oxygen, and sulfur, and optionally is substituted with —R⁴⁴ on a carbon or a nitrogen, on nitrogen by —(C(R⁴⁹)₂)ₙX⁴⁰R⁴⁴ or —C(R⁴⁹)₂)ₙNZ″Z′″, or on carbon by —(C(R⁴⁹)₂)_{q}X⁴⁰R⁴⁴ or —(C(R⁴⁹)₂)_{q}NZ″Z′″;
Z″ and Z′″ independently are H, a C₁₋₆ alkyl group, a C₂₋₆ alkenyl group, a C₂₋₆ alkynyl group, a C₆₋₁₄ aryl group, or a 5-14 membered heteroaryl group; or
Z″ and Z′″ taken together with the nitrogen to which they are attached form a 3-14 membered heterocyclic ring which optionally has an additional heteroatom selected from nitrogen, oxygen, and sulfur; and
J is fluoro, chloro, bromo, or iodo;
or a pharmaceutically acceptable salt thereof.

In certain embodiments, the method can include converting the compound of formula VII″ into a compound of formula XII′: 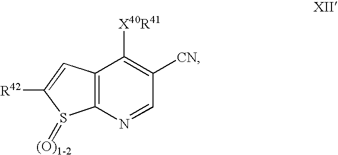
wherein R⁴¹-R⁴² and X⁴⁰ are as defined herein.

In some embodiments, X⁴⁰ can be —NH—, —NR⁴⁴—, or —NHCH₂—. In particular embodiments, X⁴⁰ can be —NH—.

In some embodiments, R⁴¹ can be a phenyl group optionally substituted with one to four substituents independently selected from -J, —CF₃, —OCF₃, —R⁴⁴, —OR⁴⁴, and —Y⁴⁰R⁴⁷; where R⁴⁷ can be a C₆₋₁₄ aryl group or a 5-14 membered heteroaryl group, each of which can be optionally substituted with one to four substituents independently selected from -J, —CF₃, —OCF₃, —R⁴⁴, and —OR⁴⁴. In certain embodiments, R⁴¹ can be a phenyl group optionally substituted with one to four substituents independently selected from —Cl, —R⁴⁴, and —OR⁴⁴. In particular embodiments, R⁴⁴ can be a C₁₋₆ alkyl group.

In some embodiments, R⁴² can be a C₆₋₁₄ aryl group or a 5-14 membered heteroaryl group, each of which can be optionally substituted with one or more —C(R⁴⁹)₂)_{q}Q. In certain embodiments, q can be 1 to 3. In particular embodiments, R⁴⁹ can be H.

In some embodiments, R⁴² can be R⁴³ where R⁴³ can be a C₂₋₆ alkynyl group, a C₆₋₁₄ aryl group, or a 5-14 membered heteroaryl group. In some embodiments, R⁴² can be optionally substituted with one or more groups independently selected from —R⁴⁸, —(CH₂)_{q}OR⁴⁸, —(CH₂)_{q}NHR⁴⁸, —(CH₂)_{q}NR⁴⁴R⁴⁸, —(CH₂)_{q}Q, —O(CH₂)ₙOR⁴⁸, —NH(CH₂)ₙOR⁴⁸, —NR⁴⁴(CH₂)ₙOR⁴⁸, —O(CH₂)ₙNHR⁴⁸, —NH(CH₂)ₙNHR⁴⁸, —NR⁴⁴(CH₂)ₙNHR⁴⁸, —O(CH₂)ₙNR⁴⁴R⁴⁸, —NH(CH₂)ₙNR⁴⁴R⁴⁸, —NR⁴⁴(CH₂)ₙNR⁴⁴R⁴⁸, —O(CH₂)ₙQ, —NH(CH₂)ₙQ, —NR⁴⁴(CH₂)ₙQ, —O(CH₂)_{q}R⁴⁸, —NH(CH₂)_{q}R⁴⁸, and —NR⁴⁴(CH₂)_{q}R⁴⁸. For example, R⁴⁴ can be H or a C₁₋₆ alkyl group. For example, R⁴⁸ can be H, a C₁₋₆ alkyl group, a C₂₋₆ cis-alkenyl group, a C₂₋₆ trans-alkenyl group, a C₂₋₆ alkynyl group, a C₆₋₁₄ aryl group, or a 5-14 membered heteroaryl group.

In some embodiments, Y⁴⁰ can be —C(O)—, —C(O)O—, —OC(O)—, —C(O)NH—, —NHC(O)—, —NHSO₂—, —SO₂NH—, —S—, —O—, or —NR⁴⁴—.

In some embodiments, Q can be NZZ′ and Z and Z′ can be the same or different. In certain embodiments, Z and Z′ can be selected from H, a C₁₋₆ alkyl group, a C₂₋₆ cis-alkenyl group, a C₂₋₆ trans-alkenyl group, a C₂₋₆ alkynyl group, a C₆₋₁₄ aryl group, and a 5-14 membered heteroaryl group; or Z and Z′ taken together with the nitrogen to which they are attached can form a 3-14 membered heterocyclic ring which can have an additional heteroatom selected from nitrogen, oxygen, and sulfur, and can be optionally substituted with —R⁴⁴ on a carbon or a nitrogen, on nitrogen by a group selected from —(CH₂)ₙOR⁴³, —(CH₂)ₙNHR⁴³, —(CH₂)ₙNR⁴⁴R⁴³, and —(CH₂)ₙNZ″Z′″, or on carbon by a group selected from —(CH₂)_{q}OR⁴³—CH₂)_{q}NHR⁴³, —(CH₂)_{q}NR⁴⁴R⁴³ and —(CH₂)_{q}NZ″Z′″. For example, Z″ and Z′″can be the same or different and each can be selected from H and a C₁₋₆ alkyl group; or Z″ and Z′″taken together with the nitrogen to which they are attached can form a 3-14 membered heterocyclic ring which can contain an additional heteroatom selected from nitrogen, oxygen, and sulfur. In certain embodiments, Q can be NZZ′ where Z and Z′ can be the same or different and can independently be H or a C₁₋₆ alkyl group. In certain embodiments, Z and Z′ taken together with the nitrogen to which they are attached can form a 3-14 membered heterocyclic ring which can have an additional heteroatom selected from nitrogen and oxygen and can be substituted on nitrogen or carbon by R⁴⁴ or on carbon by —(CH₂)₂OH.

Compounds of the present teachings can be prepared in accordance with the procedures outlined in the schemes below, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field. It will be appreciated that where typical or preferred process conditions (i.e., reaction temperatures, times, mole ratios of reactants, solvents, pressures, etc.) are given, other process conditions can also be used unless otherwise stated. Optimum reaction conditions can vary with the particular reactants or solvent used, but such conditions can be determined by one skilled in the art by routine optimization procedures. Those skilled in the art of organic synthesis will recognize that the nature and order of the synthetic steps presented can be varied for the purpose of optimizing the formation of the compounds described herein.

Preparation of compounds can involve the protection and deprotection of various chemical groups. The need for protection and deprotection and the selection of appropriate protecting groups can be readily determined by one skilled in the art. The chemistry of protecting groups can be found, for example, in Greene, et al., Protective Groups in Organic Synthesis, 4th Ed., Wiley & Sons, 2006, the entire disclosure of which is incorporated by reference herein for all purposes.

The processes described herein can be monitored according to any suitable methods known in the art. For example, product formation can be monitored by spectroscopic means, such as nuclear magnetic resonance spectroscopy (NMR, e.g., ¹H or ¹³C), infrared spectroscopy (IR), spectrophotometry (e.g., UV-visible), mass spectrometry (MS), or by chromatography such as high-performance liquid chromatography (HPLC), gas chromatography (GC), or thin layer chromatography (TLC).

The reactions or the processes described herein can be carried out in suitable solvents which can be readily selected by one skilled in the art of organic synthesis. Suitable solvents typically are substantially nonreactive with the reactants, intermediates, and/or products at the temperatures at which the reactions are carried out, i.e., temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected.

In general, compounds of formula VI or tautomers thereof can be prepared from precursor compounds according to Scheme 1 below (R³, which is H, is not shown):

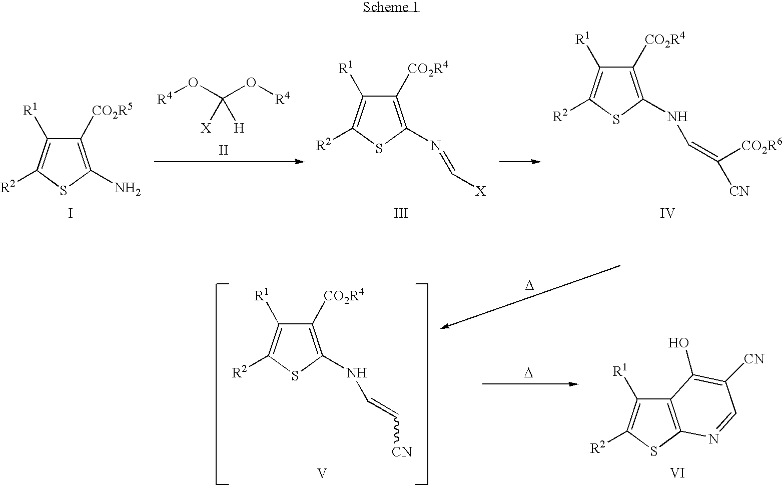

Compounds of formula I, where R⁵ can be a C₁₋₆ alkyl group and R¹ and R² are as defined herein, are commercially available or can be prepared by the Gewald reaction illustrated below: 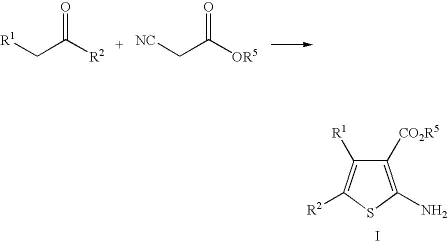
where a ketone or aldehyde can be reacted with an α-cyanoester in the presence of elemental sulfur and a base to provide the optionally substituted 2-aminothiophene-3-carboxylic acid or ester I.

The optionally substituted 2-aminothiophene-3-carboxylic acid or ester I can be treated with a compound of formula II to provide a compound of formula III, where R¹, R², R⁴, R⁵, and X are as defined herein. Depending on the reaction conditions, the carboxylate group of compound I may or may not incorporate the —OR⁴ group of compound II. Accordingly, the carboxylate group (CO₂R⁴ as shown) of compounds III, IV, and V can be either CO₂R⁴ or CO₂R⁵.

In some embodiments, compound II can be an orthoester such as, without limitation, a triethyl orthoformate or a trimethyl orthoacetate. In other embodiments, compound II can be an amide equivalent of an orthoester such as, without limitation, a dimethylformamide dimethyl acetal or a dimethylformamide diethyl acetal. In some embodiments, compound I can be reacted with between about 1 equivalent and about 10 equivalents of compound II.

In some embodiments, the reaction of the optionally substituted 2-aminothiophene-3-carboxylic acid or ester I with the compound II can be conducted neat. In other embodiments, the reaction can be performed in a suitable anhydrous solvent such as, without limitation, tetrahydrofuran, toluene, or tert-butanol.

In some embodiments, the reaction can be conducted at a temperature between about 50° C. and about 135° C. In particular embodiments, the reaction can be conducted at a temperature of about 50° C., at a temperature of about 55° C., at a temperature of about 60° C., at a temperature of about 65° C., at a temperature of about 70° C., at a temperature of about 75° C., at a temperature of about 80° C., at a temperature of about 85° C., at a temperature of about 90° C., at a temperature of about 95° C., at a temperature of about 100° C., at a temperature of about 105° C., at a temperature of about 110° C., at a temperature of about 115° C., at a temperature of about 120° C., at a temperature of about 125° C., at a temperature of about 130° C., or at a temperature of about 135° C.

After removal of any excess reagent and solvent, the reaction product III generally can be obtained as an oil, which typically can be of sufficient purity for use in the subsequent reaction without further purification.

Compound III can be treated with an α-cyano ester such as tert-butyl cyanoacetate to form a compound of formula IV, where R¹, R², R⁴, R⁶, and X are as defined herein. In certain embodiments, compound III can be treated with from about 1.5 equivalents to about 2.5 equivalents of the α-cyano ester. In particular embodiments, compound III can be reacted with about 2.0 equivalents of the α-cyano ester. In certain embodiments, compound III can be treated with from about 1.5 equivalents to about 2.5 equivalents, e.g., about 2.0 equivalents, of tert-butyl cyanoacetate.

The treatment of compound III with the α-cyano ester can be performed in various solvents, such as, without limitation, tetrahydrofuran, acetonitrile, toluene, dichloromethane, tert-butanol, or a mixture thereof. In some embodiments, this reaction can be performed in tert-butanol or a solvent including tert-butanol.

The reaction temperature can be between about 18° C. and about 110° C. In certain embodiments, the reaction can be conducted at a temperature of about 18° C., at a temperature of about 20° C., at a temperature of about 22° C., at a temperature of about 25° C., at a temperature of about 30° C., at a temperature of about 35° C., at a temperature of about 40° C., at a temperature of about 45° C., at a temperature of about 50° C., at a temperature of about 60° C., at a temperature of about 70° C., at a temperature of about 80° C., at a temperature of about 90° C., at a temperature of about 100° C., or at a temperature of about 110° C. In certain embodiments, the reaction can be performed at room temperature, for example, at about 20-30° C., for an appropriate amount of time. For example, the reaction can be performed for any period of time from about 1 hour to about 10 days.

After concentration, washing, and/or filtration, compound IV can be collected as a solid, which can be optionally purified by chromatography or recrystallization.

Compound IV then can be converted to compound VI in a thermally catalyzed reaction. Comparing to prior procedures, the conversion of compound IV to compound VI according to the present teachings is mainly driven by heat as opposed to other catalysts. For example, the reaction to provide compound VI from compound IV can be carried out in the absence of an acid or a base.

Without wishing to be bound to any particular theory, it is believed that the decarboxylation of compound IV and the intramolecular cyclization of the cyanoacrylate group of compound V can occur at about the same time. However, it is also possible, at least to some extent, that the decarboxylation and the intramolecular cyclization reactions take place sequentially (i.e., via a 2-step mechanism). Regardless, it should be understood that the methods of the present teachings are not intended to be limited in any way by the possible mechanisms presented.

The decarboxylation of compound IV and the intramolecular cyclization of the cyanoacrylate group of compound V can both be thermally catalyzed. Specifically, a solution of compound IV can be heated at a first elevated temperature to induce thermal elimination and decarboxylation to provide compound V. Compound V can be heated at a second elevated temperature that can be the same as or different from the first elevated temperature to induce the intramolecular cyclization reaction to provide a compound of formula VI where R¹, R², and R⁴ are as defined herein.

In some embodiments, compound IV can be treated in a solvent or a mixture of solvents such as, without limitation, pyridine, quinoline, toluene, xylene, biphenyl, diphenyl ether, or a mixture thereof. In certain embodiments, compound IV can be dissolved in diphenyl ether or a solvent comprising diphenyl ether. In other embodiments, compound IV can be dissolved in a mixture of biphenyl and diphenyl ether. In particular embodiments, compound IV can be dissolved in a eutectic mixture comprising 26.5% of biphenyl and 73.5% of diphenyl ether.

In some embodiments, compound IV can be converted into compound VI by heating compound IV at a substantially constant elevated temperature. In certain embodiments, a solvent can be heated to an elevated temperature to which compound IV can be added. The temperature of the reaction mixture can be maintained for an appropriate amount of time, for example, about 30 minutes to about 5 hours, whereupon compound IV can be converted to compound VI.

Compound VI can be isolated by any suitable technique. In some embodiments, compound VI can be isolated by precipitation. For example, compound VI can be isolated by adding a second solvent into the reaction mixture, by cooling the reaction mixture to a reduced temperature, or a combination thereof. In certain embodiments, the reaction mixture can be cooled, for example, to about room temperature and treated with the second solvent to provide compound VI as a solid. In certain embodiments, the reaction mixture can be cooled, treated with the second solvent, and cooled further, for example, to about room temperature to provide compound VI as a solid. In some embodiments, the second solvent can be a nonpolar solvent, including, for example, pentane, hexane, heptane, cyclohexane, cycloheptane, petroleum ether, and a mixture thereof.

In some embodiments, 4-hydroxythieno[2,3-b]pyridine-5-carbonitrile VI can be used without further purification, for example, for preparing substituted thieno[2,3-b]pyridine-5-carbonitriles. In other embodiments, compound VI can be purified by one or more suitable techniques including, for example, recrystallization.

Scheme 2 below illustrates the halogenation of compound VI at the 2-position.

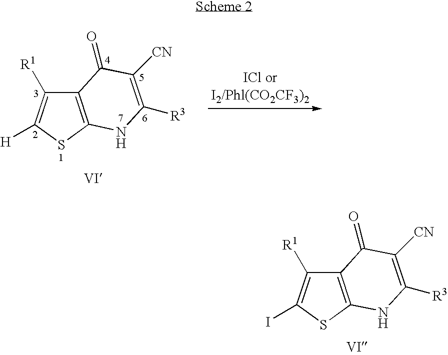

As shown, where R² is H, 4-hydroxythieno[2,3-b]pyridine-5-carbonitrile VI′ can be treated with an iodine source such as, without limitation, I₂ or ICI to effect the iodination at the 2-position. In some embodiments, ICI can be used, for example, in the form of a 1 M solution in dichloromethane or in methanol and/or in the presence of sodium acetate at room temperature. In other embodiments, I₂ can be used with or without an activating agent such as [bis(trifluoroacetoxy)iodo]benzene (PhI(CO₂CF₃)₂) in chloroform at room temperature. Use of a brominating reagent such as bromine can provide the corresponding 2-bromo-4-hydroxythieno[2,3-b]pyridine-5-carbonitrile.

Scheme 3 below illustrates the halogenation of compound VI″ at the 4-position.

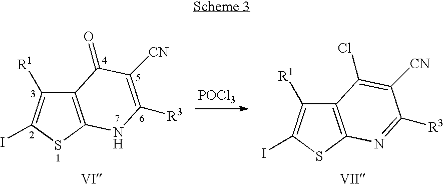

For example, treatment of 2-iodo-4-hydroxythieno[2,3-b]pyridine-5-carbonitriles VI″ with a chlorinating reagent including phosphorus oxychloride at elevated temperatures can provide the corresponding 2-iodo-4-chlorothieno[2,3-b]pyridine-5-carbonitriles VII″. Alternatively, this reaction can be carried out in phosphorus oxychloride with a catalytic amount of dimethylformamide. Procedures analogous to those described in Scheme 3 can be used for preparing 4-chlorothieno[2,3-b]pyridine-5-carbonitriles where R² is H.

Scheme 4 below illustrates the bis-halogenation of compound VII, where R¹ is H, at the 3- and 4-positions.

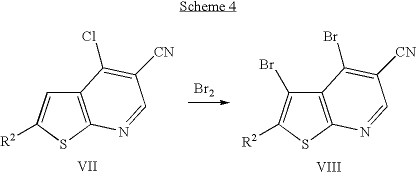

As shown in Scheme 4, compound VII, where each of R¹ and R³ is H, can be treated with bromine at elevated temperatures to provide the corresponding 3,4-dibromothieno[2,3-b]pyridine-5-carbonitrile VIII. The two bromo groups of compound VIII can be individually replaced to provide various substituted thieno[2,3-b]pyridine-5-carbonitriles which can be used as protein kinase inhibitors.

Using procedures analogous to those described in the '880 publication, the compound of formula VII″ can be converted into a compound of formula XI. Some embodiments of such conversion are illustrated in Scheme 5 below.

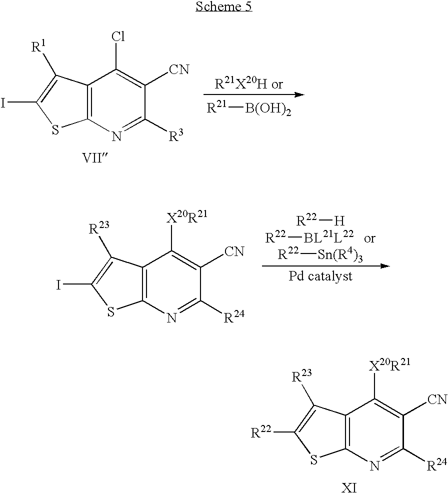

As shown in Scheme 5, compound VII″ can be treated with R²¹X²⁰H or R²¹ B(OH)₂, followed by reactions with R²²H, R²²BL²¹L²², or R²²Sn(R⁴)₃ in the presence of a Pd catalyst, to provide a compound of formula XI, where X²⁰ can be an amine, amide, —O—, or —S— linker group, each of L²¹ and L²² can be a lower alkoxy group or a hydroxy group, and R¹, R³, R⁴, R²¹R²², R²³, and R²⁴ are as defined herein.

Similarly, using procedures analogous to Scheme 5, the compound of formula VII″ can be converted into the compound of formula XI′ or formula XI″, or a pharmaceutically acceptable salt thereof.

Using procedures analogous to those described in the '880 publication and the '116 patent, the compound of formula VII″ can be converted into the compound of formula XII. In some embodiments, procedures analogous to those illustrated in Scheme 5 can be used for converting the compound of formula VII″ into the compound of formula XII or formula XII′, or a pharmaceutically acceptable salt thereof.

To facilitate a further understanding of the present teachings, the following non-limiting examples are provided for illustration.

Unless stated otherwise, the analytical HPLC conditions were as follows: a Prodigy ODS3 (0.46×15 cm) column was used, the gradient was 10% acetonitrile to 90% acetonitrile with 0.01% TFA additive in water over 20 minutes, the flow rate was 1.0 mL/min, and the temperature was 40° C.

### EXAMPLE 1

### Preparation of 4-hydroxythieno[2,3-b]pyridine-5-carbonitrile

Methyl 2-aminothiophene-3-carboxylate (80 g, 510 mmol) was treated with 250 mL of dimethylformamide-dimethylacetal and the resulting mixture was heated to 100° C. After heating overnight, the reaction was cooled and concentrated to give a dark oil. Tert-butanol (450 mL) was added to the residue followed by tert-butyl cyanoacetate (132 g, 1020 mmol). The reaction was stirred for 4 days at room temperature. The resulting thick precipitate was collected by filtration and washed extensively with tert-butanol until the washings ran clear. The pale yellow solid was dried under vacuum to give 77 grams of methyl 2-{[(1Z)-3-tert-butoxy-2-cyano-3-oxoprop-1-en-1-yl]amino}thiophene-3-carboxylate (50% yield). The mother liquor yielded additional 10 grams of methyl 2-{[(1Z)-3-tert-butoxy-2-cyano-3-oxoprop-1-en-1-yl]amino}thiophene-3-carboxylate after partial concentration and standing for several days at room temperature, mp 154-157° C.; MS (ESI) m/z 306.9 (M+H).

Diphenyl ether (250 mL) was heated to a gentle reflux using a heating mantle. Nitrogen was bubbled into the diphenyl ether as it was heating to reflux and then gently blown over the top of the solvent during the course of the reaction. Methyl 2-{[(1Z)-3-tert-butoxy-2-cyano-3-oxoprop-1-en-1-yl]amino}thiophene-3-carboxylate (14 g, 45 mmol) was added in portions over a few minutes. The reaction was heated to a gentle reflux for 3 hours then cooled to room temperature. Hexane (500 mL) was added and the resultant precipitate was filtered and washed extensively with hexane. The residual diphenyl ether was removed by stirring the solid for several hours in hexane followed by filtration to give 7.25 g of 4-hydroxythieno[2,3-b]pyridine-5-carbonitrile as a dark powder (91% yield), MS (ESI) m/z 174.9 (M+H).

### EXAMPLE 2

### Preparation of 4-chloro-2-iodothieno[2,3-b]pyridine-5-carbonitrile

4-Hydroxythieno[2,3-b]pyridine-5-carbonitrile (5.0 g, 28.4 mmol) was stirred as a suspension in 500 mL of CHCl₃. To the above slurry was added sequentially [bis(trifluoroacetoxy)iodo]benzene (18.3 g, 42.6 mmol) and iodine (10.8 g, 42.6 mmol). The mixture was stirred at room temperature for 24 hours then concentrated to approximately 150 mL. The resultant solid was filtered and the solid was washed extensively with hexane until the washings ran clear. The resultant brown solid (7.9 g) was treated with phosphorus oxychloride (60 mL) and DMF (0.6 mL) and heated to 70° C. overnight. The reaction was carefully poured over ice and the product was filtered and washed with water to give 8.0 g of 4-chloro-2-iodothieno[2,3-b]pyridine-5-carbonitrile as a brown solid. The crude product was generally used directly in subsequent steps but could be further purified by column chromatography (EtOAc/hexane), MS (ESI) m/z 320.9 (M+H).

### EXAMPLE 3

### Preparation of 3-methyl-4-oxo-4,7-dihydrothieno[2,3-b]pyridine-5-carbonitrile

Following procedures analogous to those described in Example 1, ethyl 2-{[(1Z)-3-tert-butoxy-2-cyano-3-oxoprop-1-en-1-yl]amino}-4-methylthiophene-3-carboxylate was prepared from ethyl 2-amino-4-methylthiophene-3-carboxylate, mp 144° C.; MS (ESI) m/z 335; HPLC retention time=19.3 min.

Following procedures analogous to those described in Example 1, 3-methyl-4-oxo-4,7-dihydrothieno[2,3-b]pyridine-5-carbonitrile was prepared from ethyl 2-{[(1Z)-3-tert-butoxy-2-cyano-3-oxoprop-1-en-1-yl]amino}-4-methylthiophene-3-carboxylate, mp 285° C.; MS (ESI) m/z 188.9; HPLC retention time=6.2 min.

### EXAMPLE 4

### Preparation of 4-chloro-2-iodo-3-methylthieno[2,3-b]pyridine-5-carbonitrile

Following procedures analogous to those described in Example 2, 4-chloro-2-iodo-3-methylthieno[2,3-b]pyridine-5-carbonitrile was prepared from 3-methyl-4-oxo-4,7-dihydrothieno[2,3-b]pyridine-5-carbonitrile, MS (APCI) m/z 333.8; HPLC retention time=18.1 min.

### EXAMPLE 5

### Preparation of 3-isopropyl-4-oxo-4,7-dihydrothieno[2,3-b]pyridine-5-carbonitrile

Following procedures analogous to those described in Example 1, ethyl 2-{[(1Z)-3-tert-butoxy-2-cyano-3-oxoprop-1-en-1-yl]amino}-4-isopropylthiophene-3-carboxylate was prepared from ethyl 2-amino-4-isopropylthiophene-3-carboxylate, mp 93-94° C.; MS (ESI) m/z 363.3.

Following procedures analogous to those described in Example 1, 3-isopropyl-4-oxo-4,7-dihydrothieno[2,3′-b]pyridine-5-carbonitrile was prepared from ethyl 2-{[(1Z)-3-tert-butoxy-2-cyano-3-oxoprop-1-en-1-yl]amino}-4-isopropylthiophene-3-carboxylate, mp 285° C.; MS (ESI) m/z 188.9.

### EXAMPLE 6

### Preparation of 2-iodo-3-isopropyl-4-oxo-4,7-dihydrothieno[2,3-b]pyridine-5-carbonitrile

2-Iodo-3-isopropyl-4-oxo-4,7-dihydrothieno[2,3-b]pyridine-5-carbonitrile was obtained by treatment of 3-isopropyl-4-oxo-4,7-dihydrothieno[2,3-b]pyridine-5-carbonitrile with 1 M iodine monochloride in dichloromethane and sodium acetate in methanol, MS (ESI) m/z 345.1.

### EXAMPLE 7

### Preparation of 2-iodo-3-isopropylthieno[2,3-b]pyridine-5-carbonitrile

Sodium acetate (530 mg, 6.46 mmol) was added to a suspension of 3-isopropyl-4-oxo-4,7-dihydrothieno[2,3-b]pyridine-5-carbonitrile (469 mg, 1.12 mmol) in 30 mL of dichloromethane and 5 mL of methanol at room temperature. A solution of iodine monochloride in dichloromethane (1 M) was slowly added. The reaction mixture was stirred at room temperature overnight and added into a mixture of saturated aqueous sodium metabisulfite and ice. The mixture was stirred for 30 minutes and the resulting precipitates were collected and washed with water to provide 302 mg of 2-iodo-3-isopropyl-4-oxo-4,7-dihydrothieno[2,3-b]pyridine-5-carbonitrile as a light tan solid (78% yield), MS (ESI) 345.1 (M+H).

### EXAMPLE 8

### Preparation of 4-chloro-2-iodo-3-isopropylthieno[2,3-b]pyridine-5-carbonitrile

A mixture of 2-iodo-3-isopropyl-4-oxo-4,7-dihydrothieno[2,3-b]pyridine-5-carbonitrile (296 mg, 0.86 mmol) in 3 mL of phosphorus oxychloride was heated at the reflux temperature for 2 hours, cooled, and concentrated in vacuo. The resulting residue was cooled with an ice bath and a saturated aqueous sodium bicarbonate solution was added to the residue slowly. The mixture was stirred and extracted with chloroform. The combined organic layers were washed with brine, dried over magnesium sulfate, and filtered. The filtrate was concentrated in vacuo to give a residue, which was triturated with diethyl ether to provide 177 mg of 4-chloro-2-iodo-3-isopropylthieno[2,3-b]pyridine-5-carbonitrile as an off-white solid (59% yield), mp 177-179° C., MS (ESI) 363.1 (M+H).

### EXAMPLE 9

### Preparation of 2-methyl-4-oxo-4,7-dihydrothieno[2,3-b]pyridine-5-carbonitrile

Following procedures analogous to those described in Example 1, (Z)-methyl 2-(3-tert-butoxy-2-cyano-3-oxoprop-1-enylamino)-5-methylthiophene-3-carboxylate was prepared from methyl 2-amino-5-methylthiophene-3-carboxylate, MS (ESI) m/z 321.1 (M−H).

Following procedures analogous to those described in Example 1, 2-methyl-4-oxo-4,7-dihydrothieno[2,3-b]pyridine-5-carbonitrile was prepared from (Z)-methyl 2-(3-tert-butoxy-2-cyano-3-oxoprop-1-enylamino)-5-methylthiophene-3-carboxylate, HRMS (ESI) 191.0274; HPLC retention time=5.5 min.

### EXAMPLE 10

### Preparation of 4-chloro-2-methylthieno[2,3-b]pyridine-5-carbonitrile

2-Methyl-4-oxo-4,7-dihydrothieno[2,3-b]pyridine-5-carbonitrile (Example 9, 200 mg, 1.05 mmol) was heated in 1 mL phosphorus oxychloride for 1.5 hours. The reaction was concentrated to dryness and 10 mL of water was added. After sonication, the resulting solid was filtered to give 195 mg of 4-chloro-2-methylthieno[2,3-b]pyridine-5-carbonitrile as a dark solid (94% yield), mp 110-112° C.; MS (ESI) m/z 209.0 (M+H).

### EXAMPLE 11

### Preparation of 2-ethyl-4-oxo-4,7-dihydrothieno[2,3-b]pyridine-5-carbonitrile

Following procedures analogous to those described in Example 1, (Z)-ethyl 2-(3-tert-butoxy-2-cyano-3-oxoprop-1-enylamino)-5-ethylthiophene-3-carboxylate was prepared from ethyl 2-amino-5-ethylthiophene-3-carboxylate, MS (ESI) m/z 349.2 (M−H).

Following procedures analogous to those described in Example 1, 2-ethyl-4-oxo-4,7-dihydrothieno[2,3-b]pyridine-5-carbonitrile was prepared from (Z)-ethyl 2-(3-tert-butoxy-2-cyano-3-oxoprop-1-enylamino)-5-ethylthiophene-3-carboxylate, HRMS (ESI) 205.0430; HPLC retention time=7.0 min.

### EXAMPLE 12

### Preparation of 4-chloro-2-ethylthieno[2,3-b]pyridine-5-carbonitrile

Following procedures analogous to those described in Example 10, 4-chloro-2-ethylthieno[2,3-b]pyridine-5-carbonitrile was prepared from 2-ethyl-4-oxo-4,7-dihydrothieno[2,3-b]pyridine-5-carbonitrile (Example 11) in the presence of phosphorus oxychloride, MS (ESI) m/z 223.1 (M+H).

### EXAMPLE 13

### Preparation of 2,3-dimethyl-4-oxo-4,7-dihydrothieno[2,3-b]pyridine-5-carbonitrile

Following procedures analogous to those described in Example 1, (Z)-ethyl 2-(3-tert-butoxy-2-cyano-3-oxoprop-1-enylamino)-4,5-dimethylthiophene-3-carboxylate was prepared from ethyl 2-amino-4,5-dimethylthiophene-3-carboxylate, MS (ESI) m/z 349.2 (M−H).

Following procedures analogous to those described in Example 1, 2,3-dimethyl-4-oxo-4,7-dihydrothieno[2,3-b]pyridine-5-carbonitrile was prepared from (Z)-ethyl 2-(3-tert-butoxy-2-cyano-3-oxoprop-1-enylamino)-4,5-dimethylthiophene-3-carboxylate, MS (ESI) m/z 203.0 (M−H); HPLC retention time=7.5 min.

### EXAMPLE 14

### Preparation of 4-chloro-2,3-dimethylthieno[2,3-b]pyridine-5-carbonitrile

Following procedures analogous to those described in Example 10, 4-chloro-2,3-dimethylthieno[2,3-b]pyridine-5-carbonitrile was prepared from 2,3-dimethyl-4-oxo-4,7-dihydrothieno[2,3-b]pyridine-5-carbonitrile (Example 13) in the presence of phosphorus oxychloride, MS (ESI) m/z 223.1 (M+H).

### EXAMPLE 15

### Preparation of 4-oxo-2-phenyl-4,7-dihydrothieno[2,3-b]pyridine-5-carbonitrile

Following procedures analogous to those described in Example 1, (Z)-ethyl 2-(3-tert-butoxy-2-cyano-3-oxoprop-1-enylamino)-5-phenylthiophene-3-carboxylate was prepared from ethyl 2-amino-5-phenylthiophene-3-carboxylate, MS (ESI) m/z 397.2 (M−H).

Following procedures analogous to those described in Example 1, 4-oxo-2-phenyl-4,7-dihydrothieno[2,3-b]pyridine-5-carbonitrile was prepared from (Z)-ethyl 2-(3-tert-butoxy-2-cyano-3-oxoprop-1-enylamino)-5-phenylthiophene-3-carboxylate, HRMS (ESI) 253.0432; HPLC retention time=9.9 min.

### EXAMPLE 16

### Preparation of 4-chloro-2-phenylthieno[2,3-b]pyridine-5-carbonitrile

Following procedures analogous to those described in Example 10, 4-chloro-2-phenylthieno[2,3-b]pyridine-5-carbonitrile was prepared from 4-oxo-2-phenyl-4,7-dihydrothieno[2,3-b]pyridine-5-carbonitrile (Example 15) in the presence of phosphorus oxychloride, mp 202-204° C.; HRMS (ESI-FTMS) 271.00918 (M+H).

### EXAMPLE 17

### Preparation of 2-benzyl-4-oxo-4,7-dihydrothieno[2,3-b]pyridine-5-carbonitrile

Following procedures analogous to those described in Example 1, (Z)-methyl 5-benzyl-2-(3-tert-butoxy-2-cyano-3-oxoprop-1-enylamino)thiophene-3-carboxylate was prepared from methyl 2-amino-5-benzylthiophene-3-carboxylate, HRMS (ESI) 421.1193 (M+Na); HPLC retention time=14.5 min.

Following procedures analogous to those described in Example 1, 2-benzyl-4-oxo-4,7-dihydrothieno[2,3-b]pyridine-5-carbonitrile was prepared from (Z)-methyl 5-benzyl-2-(3-tert-butoxy-2-cyano-3-oxoprop-1-enylamino)thiophene-3-carboxylate, MS (ESI) m/z 267.0 (M+H), HRMS (ESI) m/z 267.0589 (M+H).

### EXAMPLE 18

### Preparation of 2-benzyl-4-chlorothieno[2,3-b]pyridine-5-carbonitrile

Following procedures analogous to those described in Example 10, 2-benzyl-4-chlorothieno[2,3-b]pyridine-5-carbonitrile was prepared from 2-benzyl-4-oxo-4,7-dihydrothieno[2,3-b]pyridine-5-carbonitrile (Example 17) in the presence of phosphorus oxychloride, MS (ESI) m/z 285.2 (M+H); —HPLC-retention time=−12.3 min (10% acetonitrile to 95% acetonitrile with 0.02% TFA additive in water over 18 minutes).

### EXAMPLE 19

### Preparation of 3-(4-fluorophenyl)-4-oxo-4,7-dihydrothieno[2,3-b]pyridine-5-carbonitrile

Following procedures analogous to those described in Example 1, (Z)-ethyl 2-(3-tert-butoxy-2-cyano-3-oxoprop-1-enylamino)-4-(4-fluorophenyl)thiophene-3-carboxylate was prepared from ethyl 2-amino-4-(4-fluorophenyl)thiophene-3-carboxylate, MS (ESI) m/z 417.0 (M+H); HPLC retention time=14.7 min.

Following procedures analogous to those described in Example 1,3-(4-fluorophenyl)-4-oxo-4,7-dihydrothieno[2,3-b]pyridine-5-carbonitrile was prepared from (Z)-ethyl 2-(3-tert-butoxy-2-cyano-3-oxoprop-1-enylamino)-4-(4-fluorophenyl)thiophene-3-carboxylate, MS (ESI) m/z 270.7 (M+H); HPLC retention time=6.1 min (10% acetonitrile to 95% acetonitrile with 0.02% TFA additive in water over 18 minutes).

### EXAMPLE 20

### Preparation of 3-(4-chlorophenyl)-4-oxo-4,7-dihydrothieno[2,3-b]pyridine-5-carbonitrile

Following procedures analogous to those described in Example 1, (Z)-ethyl 2-(3-tert-butoxy-2-cyano-3-oxoprop-1-enylamino)-4-(4-chlorophenyl)thiophene-3-carboxylate was prepared from ethyl 2-amino-4-(4-chlorophenyl)thiophene-3-carboxylate, HRMS (ESI) 433.0988 (M+H).

Following procedures analogous to those described in Example 1,3-(4-chlorophenyl)-4-oxo-4,7-dihydrothieno[2,3-b]pyridine-5-carbonitrile was prepared from (Z)-ethyl 2-(3-tert-butoxy-2-cyano-3-oxoprop-1-enylamino)-4-(4-chlorophenyl)thiophene-3-carboxylate, HRMS (ESI) 287.0046 (M+H); HPLC retention time=12.1 min.

### EXAMPLE 21

### Preparation of 3-(4-bromophenyl)-4-oxo-4,7-dihydrothieno[2,3-b]pyridine-5-carbonitrile

Following procedures analogous to those described in Example 1, (Z)-ethyl 4-(4-bromophenyl)-2-(3-tert-butoxy-2-cyano-3-oxoprop-1-enylamino)thiophene-3-carboxylate was prepared from ethyl 2-amino-4-(4-bromophenyl)thiophene-3-carboxylate, MS (ESI) m/z 478.9 (M+H); HPLC retention time 17.0 min.

Following procedures analogous to those described in Example 1,3-(4-bromophenyl)-4-oxo-4,7-dihydrothieno[2,3-b]pyridine-5-carbonitrile was prepared from (Z)-ethyl 4-(4-bromophenyl)-2-(3-tert-butoxy-2-cyano-3-oxoprop-1-enylamino)thiophene-3-carboxylate, MS (ESI) m/z 332.7 (M+H); HPLC retention time=7.52 min (10% acetonitrile to 95% acetonitrile with 0.02% TFA additive in water over 18 minutes).

### EXAMPLE 22

### Preparation of 3-(4-methoxyphenyl)-4-oxo-4,7-dihydrothieno[2,3-b]pyridine-5-carbonitrile

Following procedures analogous to those described in Example 1, (Z)-methyl 2-(3-tert-butoxy-2-cyano-3-oxoprop-1-enylamino)-4-(4-methoxyphenyl)thiophene-3-carboxylate was prepared from methyl 2-amino-4-(4-methoxyphenyl)thiophene-3-carboxylate. Chromatographic purification (EtOAc/Hex) resulted in pure product, MS (ESI) m/z 415.0 (M+H); HPLC retention time=12.5 min.

Following procedures analogous to those described in Example 1,3-(4-methoxyphenyl)-4-oxo-4,7-dihydrothieno[2,3-b]pyridine-5-carbonitrile was prepared from (Z)-methyl 2-(3-tert-butoxy-2-cyano-3-oxoprop-1-enylamino)-4-(4-methoxyphenyl)thiophene-3-carboxylate, MS (ESI) m/z 282.9 (M+H); HPLC retention time=5.73 min (10% acetonitrile to 95% acetonitrile with 0.02% TFA additive in water over 18 minutes).

### EXAMPLE 23

### Preparation of 3-(4-fluorophenyl)-2-methyl-4-oxo-4,7-dihydrothieno[2,3-b]pyridine-5-carbonitrile

Following procedures analogous to those described in Example 1, (Z)-methyl 2-(3-tert-butoxy-2-cyano-3-oxoprop-1-enylamino)-4-(4-fluorophenyl)-5-methylthiophene-3-carboxylate was prepared from methyl 2-amino-4-(4-fluorophenyl)-5-methylthiophene-3-carboxylate, MS (ESI) m/z 417.0 (M+H); HPLC retention time=15.0 min.

Following procedures analogous to those described in Example 1,3-(4-fluorophenyl)-2-methyl-4-oxo-4,7-dihydrothieno[2,3-b]pyridine-5-carbonitrile was prepared from (Z)-methyl 2-(3-tert-butoxy-2-cyano-3-oxoprop-1-enylamino)-4-(4-fluorophenyl)-5-methylthiophene-3-carboxylate, MS (ESI) m/z 284.8 (M+H); HPLC retention time=6.8 min (10% acetonitrile to 95% acetonitrile with 0.02% TFA additive in water over 18 minutes).

### EXAMPLE 24

### Preparation of 3-(furan-2-yl)-4-oxo-4,7-dihydrothieno[2,3-b]pyridine-5-carbonitrile

Following procedures analogous to those described in Example 1, (Z)-ethyl 2-(3-tert-butoxy-2-cyano-3-oxoprop-1-enylamino)-4-(furan-2-yl)thiophene-3-carboxylate was prepared from ethyl 2-amino-4-(furan-2-yl)thiophene-3-carboxylate. Chromatographic purification on silica gel column resulted in pure product, MS (ESI) m/z 388.9 (M+H); HPLC retention time=13.6 min (10% acetonitrile to 95% acetonitrile with 0.02% TFA additive in water over 18 minutes).

Following procedures analogous to those described in Example 1,3-(furan-2-yl)-4-oxo-4,7-dihydrothieno[2,3-b]pyridine-5-carbonitrile was prepared from (Z)-ethyl 2-(3-tert-butoxy-2-cyano-3-oxoprop-1-enylamino)-4-(furan-2-yl)thiophene-3-carboxylate, MS (ESI) m/z 242.7 (M+H); HPLC retention time=4.86 min (10% acetonitrile to 95% acetonitrile with 0.02% TFA additive in water over 18 minutes).

### EXAMPLE 25

### Preparation of 3,4-dibromothieno[2,3-b]pyridine-5-carbonitrile

Bromine (0.878 mL, 17.06 mmol) was added dropwise to a suspension of 4-chlorothieno[2,3-b]pyridine-5-carbonitrile (1.66 g, 8.53 mmol) in 23 mL of acetic acid. The resulting mixture was heated at 80° C. for 24 hours. Additional bromine (0.878 mL) was added and heating at 80° C. was continued. After 24 hours, additional bromine (0.878 mL) was added and heating at 80° C. was resumed for another 24 hours. The mixture was cooled to room temperature and concentrated in vacuo. The residue was cooled to 0-5° C. and neutralized with a saturated aqueous sodium bicarbonate solution and extracted with dichloromethane. The organic phase was washed twice with brine, dried over sodium sulfate, filtered, and concentrated in vacuo. The residue was purified by column chromatography eluting with a gradient of 0 to 70% dichloromethane in hexane followed by dichloromethane to provide 694 mg of 3,4-dibromothieno[2,3-b]pyridine-5-carbonitrile as a white solid, mp 204-206° C., MS 315.8 (M−H)⁻. Additional fractions provided 831 mg of a mixture of 3,4-dibromothieno[2,3-b]pyridine-5-carbonitrile and 3-bromo-4-chlorothieno[2,3-b]pyridine-5-carbonitrile.

### EXAMPLE 26

### Preparation of 2-methyl-4-(4-methyl-1H-indol-5-ylamino)thieno[2,3-b]pyridine-5-carbonitrile

4-Chloro-2-methylthieno[2,3-b]pyridine-5-carbonitrile (Example 10, 80 mg, 0.38 mmol) was treated with 5-amino-4-methylindole (111 mg, 0.76 mmol) in 3 mL of ethanol. After heating at 90° C. for 14 hours in a sealed vial, the reaction was cooled and treated with 2 mL of water. The resulting precipitate was filtered and washed with ethanol to give 43 mg of 2-methyl-4-(4-methyl-1H-indol-5-ylamino)thieno[2,3-b]pyridine-5-carbonitrile as a brown solid (36% yield), HRMS (ESI) 319.1015 (M+H); HPLC retention time=13.4 min.

### EXAMPLE 27

### Preparation of 2-ethyl-4-(4-methyl-1H-indol-5-ylamino)thieno[2,3-b]pyridine-5-carbonitrile

Following procedures analogous to those described in Example 26, 2-ethyl-4-(4-methyl-1H-indol-5-ylamino)thieno[2,3-b]pyridine-5-carbonitrile was prepared from 4-chloro-2-ethylthieno[2,3-b]pyridine-5-carbonitrile (Example 12), HRMS (ESI) 333.1168 (M+H); HPLC retention time=14.7 min.

### EXAMPLE 28

### Preparation of 4-(4-methyl-1H-indol-5-ylamino)-2-phenylthieno[2,3-b]pyridine-5-carbonitrile

Following procedures analogous to those described in Example 26, 4-(4-methyl-1H-indol-5-ylamino)-2-phenylthieno[2,3-b]pyridine-5-carbonitrile was prepared from 4-chloro-2-phenylthieno[2,3-b]pyridine-5-carbonitrile (Example 16), HRMS (ESI-FTMS) 381.1171 (M+H); HPLC retention time=16.8 min.

### EXAMPLE 29

### Preparation of 2-benzyl-4-(4-methyl-1H-indol-5-ylamino)thieno[2,3-b]pyridine-5-carbonitrile

Following procedures analogous to those described in Example 26, 2-benzyl-4-(4-methyl-1H-indol-5-ylamino)thieno[2,3-b]pyridine-5-carbonitrile was prepared from 2-benzyl-4-chlorothieno[2,3-b]pyridine-5-carbonitrile (Example 18). Purification by HPLC resulted in pure product, MS (ESI) m/z 394.1 (M+H).

### EXAMPLE 30

### Preparation of 2-bromo-4-hydroxythieno[2,3-b]pyridine-5-carbonitrile

Bromine (292 μL, 5.68 mmol) was added dropwise to a suspension of 4-hydroxythieno[2,3-b]pyridine-5-carbonitrile (500 mg, 2.84 mmol) in 8 mL of acetic acid. The resulting mixture was heated at 80° C. for 6 hours, cooled to room temperature, and poured into a mixture of saturated aqueous sodium bicarbonate and ice. The precipitate was collected by filtration and washed with water and diethyl ether. The solid was dried in vacuo to provide 573 mg of 2-bromo-4-hydroxythieno[2,3-b]pyridine-5-carbonitrile as a brown solid (79% yield), mp>255° C.; MS (ESI) m/z 252.9 (M−H); HRMS (ESI) 254.92242 (M+H).

### EXAMPLE 31

### Preparation of 2-bromo-4-chlorothieno[2,3-b]pyridine-5-carbonitrile

A mixture of 2-bromo-4-hydroxythieno[2,3-b]pyridine-5-carbonitrile (500 mg, 1.96 mmol) in 2 mL of phosphorus oxychloride was heated at the reflux temperature for 2 hours. The mixture was cooled and poured into a mixture of saturated aqueous sodium bicarbonate and ice. The precipitate was collected by filtration and washed with water. The solid was dried in vacuo to give 446 mg of 2-bromo-4-chlorothieno[2,3-b]pyridine-5-carbonitrile as a brown solid (83% yield), mp 158-166° C., MS (APCI) 271.9 (M−H).

Variations, modifications, and other implementations of what is described herein will occur to those of ordinary skill in the art without departing from the spirit and the essential characteristics of the invention. Accordingly, the scope of the present teachings is to be defined not by the preceding illustrative description but instead by the following claims, and all changes that come within the meaning and range of equivalency of the claims are intended to be embraced therein.

1. A method for preparing a compound of formula VI or a tautomer thereof: 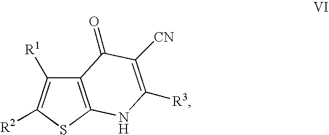
the method comprising heating a compound of formula IV: 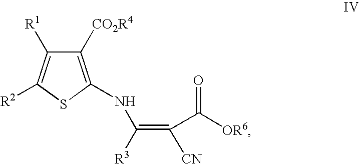
wherein:
R¹ is H, halogen, a C₁₋₆ alkyl group, a C₆₋₁₄ aryl group, a 5-14 membered heteroaryl group, a —(C₁₋₆ alkyl)-C₆₋₁₄ aryl group, or a —(C₁₋₆ alkyl)-5-14 membered heteroaryl group, wherein each of the C₆₋₁₄ aryl groups and the 5-14 membered heteroaryl groups optionally is substituted with 1-4 groups independently selected from a halogen, a C₁₋₆ alkyl group, and a C₁₋₆ alkoxy group;
R² is H, halogen, a C₁₋₆ alkyl group, a C₆₋₁₄ aryl group, a 5-14 membered heteroaryl group, a —(C₁₋₆ alkyl)-C₆₋₁₄ aryl group, or a —(C₁₋₆ alkyl)-5-14 membered heteroaryl group, wherein each of the C₆₋₁₄ aryl groups and the 5-14 membered heteroaryl groups optionally is substituted with 1-4 groups independently selected from a halogen, a C₁₋₆ alkyl group, and a C₁₋₆ alkoxy group;
R³ is H;
R⁴ is a C₁₋₆ alkyl group; and
R⁶ is a group capable of forming a carbocation.

2. The method of claim 1, wherein R¹ is selected from H, Br, I, a methyl group, an ethyl group, an isopropyl group, a phenyl group, a 4-fluorophenyl group, a 4-chlorophenyl group, a 4-bromophenyl group, a 4-methoxyphenyl group, a benzyl group, and a furanyl group.

3. The method of claim 1, wherein R² is selected from H, Br, I, a methyl group, an ethyl group, an isopropyl group, a benzyl group, a phenyl group, a 4-fluorophenyl group, a 4-chlorophenyl group, a 4-bromophenyl group, a 4-methoxyphenyl group, and a furanyl group.

4. The method of claim 1, wherein R⁶ is a tertiary alkyl group.

5. The method of claim 4, wherein R⁶ is a tert-butyl group.

6. The method of claim 1, comprising heating the compound of formula IV in a solvent at a temperature between about 200° C. and about 300° C.

7. The method of claim 6, comprising heating the solvent and adding the compound of formula IV into the heated solvent.

8. The method of claim 1, comprising heating a solvent at a first elevated temperature, adding the compound of formula IV into the heated solvent, and heating the compound of formula IV at a second elevated temperature.

9. The method of claim 8, wherein the first elevated temperature and the second elevated temperature are the same.

10. The method of claim 8, wherein the second elevated temperature is different from the first elevated temperature.

11. The method of claim 8, wherein each of the first elevated temperature and the second elevated temperature is independently between about 200° C. and about 300° C.

12. The method of claim 11, wherein the first elevated temperature is between about 200° C. and about 260° C.

13. The method of claim 11, wherein the second elevated temperature is between about 250° C. and about 260° C.

14. The method of claim 6, wherein the solvent has a boiling temperature of greater than or equal to about 200° C.

15. The method of claim 6, wherein the solvent comprises diphenyl ether or biphenyl.

16. The method of claim 15, wherein the solvent is selected from diphenyl ether, biphenyl, and a mixture thereof.

17. The method of claim 1, wherein the compound of formula IV is prepared by reacting a compound of formula III: 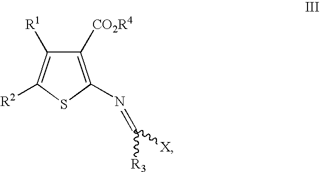
with an α-cyanoester, wherein X is —OR⁴ or —NR⁴R⁴.

18. The method of claim 17, wherein the reaction of the compound of formula III with the α-cyanoester is performed in a solvent comprising tert-butanol.

19. The method of claim 17, wherein the reaction of the compound of formula III with the α-cyanoester is performed at room temperature.

20. The method of claim 17, wherein the α-cyanoester is tert-butyl cyanoacetate.

21. The method of claim 17, wherein the compound of formula III is prepared by reacting a compound of formula I: 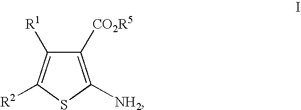
with a compound of formula II: 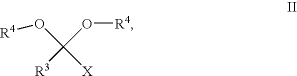
wherein R⁵ is a C₁₋₆ alkyl group.

22. The method of claim 21, wherein the compound of formula II is dimethylformamide dimethyl acetal or dimethylformamide diethyl acetal.

23. The method of claim 1, wherein the compound of formula VI is a compound of formula VI′: 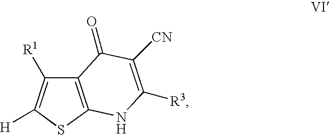
further comprising treating the compound of formula VI′ with an iodine source to form a compound of formula VI″:

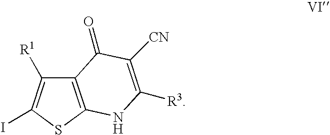

24. The method of claim 23, wherein the iodine source is I₂ or ICI.

25. The method of claim 1, further comprising treating the compound of formula VI with a chlorinating reagent to provide a compound of formula VII:

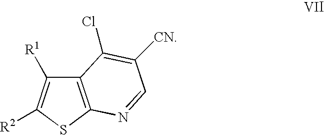

26. The method of claim 25, comprising reacting the compound of formulas VI, wherein R² is I, with a chlorinating reagent to provide a compound of formula VII″:

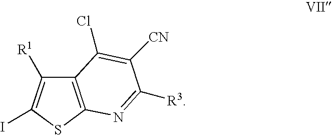

27. The method of claim 25, wherein the chlorinating reagent is phosphorus oxychloride or thionyl chloride.

28. The method of claim 25, further comprising converting the compound of formula VII, wherein R¹ is H, to a compound of formula VIII:

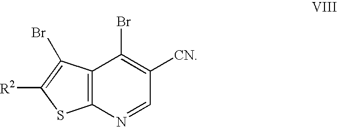

29. The method of claim 26, further comprising converting the compound of formula VII″ into a compound of formula XI: 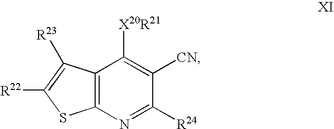
wherein:
X²⁰ is a) —NR²⁵—Y²⁰—, b) —O—Y²⁰—, c) —S(O)ₘ—Y²⁰—, d) —S(O)ₘNR²⁵—Y²⁰—, e) —NR²⁵S(O)ₘ—Y²⁰—, f) —C(O)NR²⁵—Y²⁰—, g) —NR²⁵C(O)—Y²⁰—, h) —C(S)NR²⁵—Y²⁰—, i) —NR²⁵C(S)—Y²⁰ j) —C(O)O—Y²⁰—, k) —OC(O)—Y²⁰—, l) —C(O)—Y²⁰—, or m) a covalent bond;
Y²⁰, at each occurrence, is a) a divalent C₁₋₁₀ alkyl group, b) a divalent C₂₋₁₀ alkenyl group, c) a divalent C₂₋₁₀ alkynyl group, d) a divalent C₁₋₁₀ haloalkyl group, or e) a covalent bond;
R²¹ is a) a C₁₋₁₀ alkyl group, b) a C₃₋₁₀ cycloalkyl group, c) a 3-12 membered cycloheteroalkyl group, d) a C₆₋₁₄ aryl group, or e) a 5-13 membered heteroaryl group, wherein each of a)-e) optionally is substituted with 1-4 R²⁶ groups;
R²² is a) H, b) halogen, c) —C(O)R²⁸, d) —C(O)OR²⁸, e) —C(O)NR²⁹R³⁰, f) —C(S)R²⁸ g) —C(S)OR²⁸, h) —C(S)NR²⁹R³⁰, i) a C₁₋₁₀ alkyl group, j) a C₂₋₁₀ alkenyl group, k) a C₂₋₁₀ alkynyl group, l) a C₃₋₁₀ cycloalkyl group, m) a C₆₋₁₄ aryl group, n) a 3-12 membered cycloheteroalkyl group, or o) a 5-13 membered heteroaryl group, wherein each of i)-o) optionally is substituted with 1-4 R²⁶ groups;
R²³ is a) H, b) halogen, c) —OR²⁸, d) —NR²⁹R³⁰, e) —N(O)R²⁹R³⁰, f) —S(O)ₘR²⁸, g) —S(O)ₘOR²⁸, h) —C(O)R²⁸, i) —C(O)OR²⁸, j) —C(O)NR²⁹R³⁰, k) —C(S)R²⁸, l) —C(S)OR²⁸, m) —C(S)NR²⁹R³⁰, n) —Si(C₁₋₁₀ alkyl group)₃, o) a C₁₋₁₀ alkyl group, p) a C₂₋₁₀ alkenyl group, q) a C₂₋₁₀ alkynyl group, r) a C₃₋₁₀ cycloalkyl group, s) a C₆₋₁₄ aryl group, t) a 3-12 membered cycloheteroalkyl group, or u) a 5-13 membered heteroaryl group, wherein each of o)-u) optionally is substituted with 1-4 R²⁶ groups;
R²⁴ is a) H, b) halogen, c) a C₁₋₁₀ alkyl group, d) a C₂₋₁₀ alkenyl group, e) a C₂₋₁₀ alkynyl group, f) a C₁₋₁₀haloalkyl group, g) a C₃₋₁₀ cycloalkyl group, h) a C₆₋₁₄ aryl group, i) a 3-12 membered cycloheteroalkyl group, or j) a 5-13 membered heteroaryl group, wherein each of c)-j) optionally is substituted with 1-4 R²⁶ groups;
R²⁵, at each occurrence, is a) H, b) a C₁₋₁₀ alkyl group, c) a C₂₋₁₀ alkenyl group, d) a C₂₋₁₀ alkynyl group, or e) a C₁₋₁₀ haloalkyl group;
R²⁶, at each occurrence, is a) R²⁷ or b) —Y²⁰—R²⁷;
R²⁷, at each occurrence, is a) halogen, b) —CN, c) —NO₂, d) oxo, e) —OR²⁸, f) —NR²⁹R³⁰ g) —N(O)R²⁹R³⁰, h) —S(O)ₘR²⁸, i) —S(O)ₘOR²⁸, j) —SO₂NR²⁹R³⁰, k) —C(O)R²⁸, l) —C(O)OR²⁸, m) —C(O)NR²⁹R³⁰, n) —C(S)R²⁸, o) —C(S)OR²⁸, p) —C(S)NR²⁹R³⁰, q) —Si(C₁₋₁₀ alkyl)₃, r) a C₁₋₁₀ alkyl group, s) a C₂₋₁₀ alkenyl group, t) a C₂₋₁₀ alkynyl group, u) a C₁₋₁₀ haloalkyl group, v) a C₃₋₁₀ cycloalkyl group, w) a C₆₋₁₄ aryl group, x) a 3-12 membered cycloheteroalkyl group, or y) a 5-13 membered heteroaryl group, wherein each of r)-y) optionally is substituted with 1-4 R³¹ groups;
R²⁸, at each occurrence, is a) H, b) —C(O)R³⁴, c) —C(O)OR³⁴, d) a C₁₋₁₀ alkyl group, e) a C₂₋₁₀ alkenyl group, f) a C₂₋₁₀ alkynyl group, g) a C₁₋₁₀ haloalkyl group, h) a C₃₋₁₀ cycloalkyl group, i) a C₆₋₁₄ aryl group, j) a 3-12 membered cycloheteroalkyl group, or k) a 5-13 membered heteroaryl group, wherein each of d)-k) optionally is substituted with 1-4 R³¹ groups;
R²⁹ and R³⁰, at each occurrence, independently are a) H, b) —OR³³, c) —NR³⁴R³⁵, d) —S(O)ₘR³⁴, e) —S(O)ₘOR³⁴, f) —S(O)₂NR³⁴R³⁵, g) —C(O)R³⁴, h) —C(O)OR³⁴, i) —C(O)NR³⁴R³⁵, j) —C(S)R³⁴, k) —C(S)OR³⁴, l) —C(S)NR³⁴R³⁵, m) a C₁₋₁₀ alkyl group, n) a C₂₋₁₀ alkenyl group, o) a C₂₋₁₀ alkynyl group, p) a C₁₋₁₀ haloalkyl group, q) a C₃₋₁₀ cycloalkyl group, r) a C₆₋₁₄ aryl group, s) a 3-12 membered cycloheteroalkyl group, or t) a 5-13 membered heteroaryl group, wherein each of m)-t) optionally is substituted with 1-4 R³¹ groups;
R³¹, at each occurrence, is a) R³² or b) —Y²⁰—R³²;
R³², at each occurrence, is a) halogen, b) —CN, c) —NO₂, d) oxo, e) —OR³³, f) —NR³⁴R³⁵, g) —N(O)R³⁴R³⁵, h) —S(O)ₘR³³, i) —S(O)ₘOR³³, j) —SO₂NR³⁴R³⁵ k) —C(O)R³³, l) —C(O)OR³³, m) —C(O)NR³⁴R³⁵, n) —C(S)R³³, o) —C(S)OR³³, p) —C(S)NR³⁴R³⁵, q) —Si(C₁₋₁₀ alkyl)₃, r) a C₁₋₁₀ alkyl group, s) a C₂₋₁₀ alkenyl group, t) a C₂₋₁₀ alkynyl group, u) a C₁₋₁₀ haloalkyl group, v) a C₃₋₁₀ cycloalkyl group, w) a C₆₋₁₄ aryl group, x) a 3-12 membered cycloheteroalkyl group, or y) a 5-13 membered heteroaryl group, wherein each of r)-y) optionally is substituted with 1-4 R³⁶ groups;
R³³, at each occurrence, is selected from a) H, b) —C(O)R³⁴, c) —C(O)OR³⁴, d) a C₁₋₁₀ alkyl group, e) a C₂₋₁₀ alkenyl group, f) a C₂₋₁₀ alkynyl group, g) a C₁₋₁₀ haloalkyl group, h) a C₃₋₁₀ cycloalkyl group, i) a C₆₋₁₄ aryl group, j) a 3-12 membered cycloheteroalkyl group, and k) a 5-13 membered heteroaryl group, wherein each of d)-k) optionally is substituted with 1-4 R³⁶ groups;
R³⁴ and R³⁵, at each occurrence, independently are a) H, b) a C₁₋₁₀ alkyl group, c) a C₂₋₁₀ alkenyl group, d) a C₂₋₁₀ alkynyl group, e) a C₁₋₁₀ haloalkyl group, f) a C₃₋₁₀ cycloalkyl group, g) a C₆₋₁₄ aryl group, h) a 3-12 membered cycloheteroalkyl group, or i) a 5-13 membered heteroaryl group, wherein each of b)-i) optionally is substituted with 1-4 R³⁶ groups;
R³⁶, at each occurrence, is a) halogen, b) —CN, c) —NO₂, d) —OH, e) —NH₂, f) —NH(C₁₋₁₀ alkyl), g) oxo, h) —N(C₁₋₁₀ alkyl)₂, i) —SH, j) —S(O)ₘ—C₁₋₁₀ alkyl, k) —S(O)₂OH, l) —S(O)ₘ—OC₁₀ alkyl, m) —C(O)—C₁₋₁₀ alkyl, n) —C(O)OH, o) —C(O)—OC₁₋₁₀ alkyl, p) —C(O)NH₂, q) —C(O)NH—C₁₋₁₀ alkyl, r) —C(O)N(C₁₋₁₀ alkyl)₂, s) —C(S)NH₂, t) —C(S)NH—C₁₋₁₀ alkyl, u) —C(S)N(C₁₋₁₀ alkyl)₂, v) a C₁₋₁₀ alkyl group, w) a C₂₋₁₀ alkenyl group, x) a C₂₋₁₀ alkynyl group, y) a C₁₋₁₀ alkoxy group, z) a C₁₋₁₀ haloalkyl group, aa) a C₃₋₁₀ cycloalkyl group, ab) a C₆₋₁₄ aryl group, ac) a 3-12 membered cycloheteroalkyl group, or ad) a 5-13 membered heteroaryl group; and
m is 0, 1, or 2;
or a pharmaceutically acceptable salt thereof.

30. The method of claim 26, further comprising converting the compound of formula VII″ into a compound of formula XII: 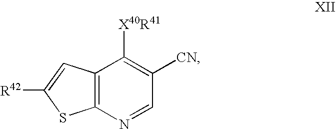
wherein:
X⁴⁰ is —NH—, —NR⁴⁴—, —O—, —S(O)ₘ—, or —NHCH₂—;
m is 0, 1, or 2;
n is 2, 3, 4, or 5;
q is 0, 1, 2, 3, 4, or 5;
R⁴¹ is a phenyl ring optionally substituted with one to four substituents independently selected from -J, —NO₂, —CN, —N₃, —CHO, —CF₃, —OCF₃, —R⁴⁴, —OR⁴⁴, —S(O)ₘR⁴⁴, —NR⁴⁴R⁴⁴, —NR⁴⁴S(O)ₘR⁴⁴, —OR⁴⁶OR⁴⁴, —OR⁴⁶NR⁴⁴R⁴⁴, —N(R⁴⁴)R⁴⁶OR⁴⁴, —N(R⁴⁴)R⁴⁶NR⁴⁴R⁴⁴, —NR⁴⁴C(O)R⁴⁴, —C(O)R⁴⁴, —C(O)OR⁴⁴, —C(O)NR⁴⁴R⁴⁴, —OC(O)R⁴⁴, —OC(O)OR⁴⁴, —OC(O)NR⁴⁴R⁴⁴, —NR⁴⁴C(O)R⁴⁴, —NR⁴⁴C(O)OR⁴⁴, —NR⁴⁴C(O)NR⁴⁴R⁴⁴, —R⁴⁵OR⁴⁴, —R⁴⁵NR⁴⁴R⁴⁴, —R⁴⁵S(O)ₘR⁴⁴, —R⁴⁵C(O)R⁴⁴, —R⁴⁵C(O)OR⁴⁴, —R⁴⁵C(O)NR⁴⁴R⁴⁴, —R⁴⁵OC(O)R⁴⁴-R⁴⁵OC(O)OR⁴⁴, —R⁴⁵OC(O)NR⁴⁴R⁴⁴, —R⁴⁵NR⁴⁴C(O)R⁴⁴, —R⁴⁵NR⁴⁴C(O)OR⁴⁴, —R⁴⁵NR⁴⁴C(O)NR⁴⁴R⁴⁴, and —Y⁴⁰R⁴⁷;
R⁴² is —H, —R⁴³, -J, —C(O)X⁴⁰R⁴³, or —CHO;
R⁴³ is a C₁₋₆ alkyl group, a C₂₋₆ cis-alkenyl group, a C₂₋₆ trans-alkenyl group, a C₂₋₆ alkynyl group, a C₆₋₁₄ aryl group, or a 5-14 membered heteroaryl group, each of which optionally is substituted by one or more groups selected from —C(O)X⁴⁰R⁴⁸, —CHO, —C(O)Q, 1,3-dioxolane, —R⁴⁸—(C(R⁴⁹)₂)_{q}X⁴⁰R⁴⁸, —C(R⁴⁹)₂)_{q}Q, —X⁴⁰(C(R⁴⁹)₂)ₙX⁴⁰R⁴⁸, —X⁴⁰(C(R⁴⁹)₂)ₙQ, and —X⁴⁰(C(R⁴⁹)₂)_{q}R⁴⁸;
R⁴⁴ is H, a C₁₋₆ alkyl group, a C₂₋₆ cis-alkenyl group, a C₂₋₆ trans-alkenyl group, or a C₂₋₆ alkynyl group;
R⁴⁵ is a divalent group selected from a C₁₋₆ alkyl group, a C₂₋₆ alkenyl group, and a C₂₋₆ alkynyl group;
R⁴⁶ is a divalent C₂₋₆ alkyl group;
R⁴⁷ is a C₃₋₇ cycloalkyl group, a C₆₋₁₄ aryl group, or a 5-14 membered heteroaryl group, a C₆₋₁₄ aryl or a 5-14 membered heteroaryl fused to one to three C₆₋₁₄ aryl or 5-14 membered heteroaryl rings, wherein each of the aryl groups, the cycloalkyl group, and the heteroaryl groups optionally is substituted with one to four substituents independently selected from a C₆₋₁₄ aryl group, —CH₂—C₆₋₁₄ aryl group, —NH—C₆₋₁₄ aryl group, —O—C₆₋₁₄ aryl group, —S(O)ₘ—C₆₋₁₄ aryl group, -J, —NO₂, —CN, —N₃, —CHO, —CF₃, —OCF₃, —R⁴⁴—OR⁴⁴, —S(O)ₘR⁴⁴, —NR⁴⁴R⁴⁴, —NR⁴⁴S(O)ₘR⁴⁴, —OR⁴⁶OR⁴⁴, —OR⁴⁶NR⁴⁴R⁴⁴, —N(R⁴⁴)R⁴⁶OR⁴⁴, —N(R⁴⁴)R⁴⁶NR⁴⁴R⁴⁴, —NR⁴⁴C(O)R⁴⁴, —C(O)R⁴⁴—C(O)OR⁴⁴, —C(O)NR⁴⁴R⁴⁴, —OC(O)R⁴⁴, —OC(O)OR⁴⁴, —OC(O)NR⁴⁴R⁴⁴, —NR⁴⁴C(O)R⁴⁴, —NR⁴⁴C(O)OR⁴⁴, —NR⁴⁴C(O)NR⁴⁴R⁴⁴, —R⁴⁵OR⁴⁴, —R⁴⁵NR⁴⁴R⁴⁴, —R⁴⁵S(O)ₘR⁴⁴, —R⁴⁵C(O)R⁴⁴, —R⁴⁵C(O)OR⁴⁴, —R⁴⁵C(O)NR⁴⁴R⁴⁴, —R⁴⁵C(O)R⁴⁴, —R⁴⁵C(O)OR⁴⁴, —R⁴⁵C(O)NR⁴⁴R⁴⁴, —R⁴⁵OC(O)R⁴⁴, —R⁴⁵OC(O)OR⁴⁴, —R⁴⁵OC(O)NR⁴⁴R⁴⁴, R⁴⁵NR⁴⁴C(O)R⁴⁴, —R⁴⁵NR⁴⁴C(O)OR⁴⁴, and —R⁴⁵NR⁴⁴C(O)NR⁴⁴R⁴⁴;
R⁴⁸ is H, a C₁₋₆ alkyl group, a C₂₋₆ cis-alkenyl group, a C₂₋₆ trans-alkenyl group, a C₂₋₆ alkynyl group, a C₆₋₁₄ aryl group, or a 5-14 membered heteroaryl group;
R⁴⁹ is —R⁴⁴ or —F;
Y⁴⁰ is —C(O)—, —C(O)O—, —OC(O)—, —C(O)NH—, —NHC(O)—, —NHSO₂—, —SO₂NH—, —C(OH)H—, —X⁴⁰(C(R⁴⁹)₂)_{q}—, —(C(R⁴⁹)₂)_{q}—, —(C(R⁴⁹)₂)_{q}X⁴⁰—, —C≡C—, cis- or trans-—CH═CH—, or a divalent C₃₋₁₀ cycloalkyl group;
Q is NZZ′ wherein Z and Z′ are the same or different and are independently H, a C₁₋₆ alkyl group, a C₂₋₆ alkenyl group, a C₂₋₆ alkynyl group, a C₆₋₁₄ aryl group, or a 5-14 membered heteroaryl group; or
Z and Z′ taken together with the nitrogen to which they are attached form a 3-14 membered heterocyclic ring which optionally has an additional heteroatom selected from nitrogen, oxygen, and sulfur, and optionally is substituted with —R⁴⁴ on a carbon or a nitrogen, on nitrogen by —(C(R⁴⁹)₂)ₙX⁴⁰R⁴⁴ or —C(R⁴⁹)₂)ₙNZ″Z′″, or on carbon by —(C(R⁴⁹)₂)_{q}X⁴⁰R⁴⁴ or —(C(R⁴⁹)₂)_{q}NZ″Z′″;
Z″ and Z′″independently are H, a C₁₋₆ alkyl group, a C₂₋₆ alkenyl group, a C₂₋₆ alkynyl group, a C₆₋₁₄ aryl group, or a 5-14 membered heteroaryl group; or
Z″ and Z′″taken together with the nitrogen to which they are attached form a 3-14 membered heterocyclic ring which optionally has an additional heteroatom selected from nitrogen, oxygen, and sulfur; and
J is fluoro, chloro, bromo, or iodo;
or a pharmaceutically acceptable salt thereof.
